(19) United States
(12) Patent Application Publication
(10) Pub. No.: US 2024/0277744 A1
(43) Pub. Date: Aug. 22, 2024

(54) TREATMENT OF CHEMORESISTANT CANCER CELLS

(71) Applicant: Cornell University, Ithaca, NY (US)

(72) Inventors: Shuibing Chen, Pelham, NY (US); Todd R. Evans, New York, NY (US)

(21) Appl. No.: 18/439,716

(22) Filed: Feb. 12, 2024

Related U.S. Application Data
(60) Provisional application No. 63/444,733, filed on Feb. 10, 2023.

(51) Int. Cl. A61K 31/7048; A61P 35/00

(52) U.S. Cl. CPC A61K 31/7048; A61P 35/00

(57) ABSTRACT

Use of Cymarin or a structural analog thereof to treat cancer or a combination of cancers, any one of which or all of which are a chemoresistant cancer or chemoresistant cancers or the like. In various examples, a cancer is pancreatic cancer, colon cancer, lung cancer, breast cancer, prostate cancer, or the like, any of which may be a chemoresistant cancer, or any combination thereof. In various examples, Cymarin or a structural analog thereof potentiates a chemotherapeutic agent for the treatment of a chemoresistant cance. In various examples, a composition, which may be a pharmaceutical composition, comprises Cymarin or a structural analog thereof a combination thereof.

### CROSS REFERENCE TO RELATED APPLICATIONS

This application claims the benefit of U.S. Provisional Application No. 63/444,733, filed Feb. 10, 2023; the contents of the above-identified application are hereby fully incorporated herein by reference in their entirety.

### SEQUENCE LISTING

The instant application contains a Sequence Listing, which is submitted in .xml format and is hereby incorporated by reference in its entirety. Said .xml file is named “018617_01755_ST26.xml”, was created on Feb. 11, 2024, and is 18,680 bytes in size.

### BACKGROUND OF THE DISCLOSURE

Pancreatic cancer is one of the most challenging solid tumors, with a 5-year survival rate of 7.2%. The major treatment option for unresectable/metastatic pancreatic cancer is chemotherapy, which can be combined with surgery as neoadjuvant or adjuvant therapy. Gemcitabine (GEM), either by itself or in combination with other drugs, is considered a first-line agent for locally advanced and metastatic pancreatic cancer to improve quality of life and prolong survival. However, outcome remains poor due to a high rate of chemoresistance.

Antigen-specific targeted therapy is potentially effective to enhance treatment by specifically eliminating chemoresistant cancer cells (CCCs), as developed to treat leukemia. For example, CD126 blocking antibody enhanced chemotherapy on chemoresistant chronic lymphocytic leukemia cells, which display active signaling through the IL6/CD126/STAT3 axis. In clinical trials, Gemtuzumab ozogamicin, the conjugation of anti-CD33 antibody and calicheamicin, improved survival of acute myeloid leukemia patients. However, few antigen-specific targeted therapies have been tested to conquer chemoresistance of solid tumors, including pancreatic cancer. One of the obstacles is to identify specific cell surface marker(s) that label CCCs.

Several cell surface markers have been reported to enrich chemoresistant pancreatic cancer cells. For example, CD133+ cancer stem cells show high resistance to standard chemotherapy; CD44 sustains GEM-resistance in pancreatic cancer cells; c-MET is also enhanced in GEM-resistant pancreatic cancer cells and necessary for chemoresistance. Both chemical and antibody-based strategies have been evaluated to target cell surface markers, and some phase I clinical trials have proceeded for CD44 and c-MET in pancreatic cancer. In these trials, a major potential problem is that the markers being targeted are widely expressed in healthy tissues. For example, CD44 is highly expressed in lymphocytes, CD133 is a hematopoietic stem/progenitor cell marker, and c-Met is extensively expressed in liver, gastro-intestinal tract and kidney. Expression of these antigens in healthy tissues raises the concern of “off-target” effects in “targeted” therapy.

### SUMMARY OF THE DISCLOSURE

In an aspect, the present disclosure provides compositions, such as, for example, pharmaceutical compositions or the like, comprising Cymarin and/or a structural analog or analogs thereof and/or one or more pharmaceutically acceptable salt, a salt, a partial salt, a solvate, a hydrate, a polymorph, or a prodrug, or the like of the Cymarin and/or the structural analog(s) and/or a stereoisomer or a mixture of stereoisomers, an isotopic variant, or a tautomer, or the like of the Cymarin and/or the structural analog(s). In various examples, a composition is a pharmaceutical composition further comprising one or more pharmaceutically acceptable excipient(s) or the like.

In an aspect, the present disclosure provides methods of using Cymarin and/or a structural analog or analogs thereof and/or one or more pharmaceutically acceptable salt, a salt, a partial salt, a solvate, a hydrate, a polymorph, or a prodrug, or the like of the Cymarin and/or the structural analog(s) and/or a stereoisomer or a mixture of stereoisomers, an isotopic variant, or a tautomer, or the like of the Cymarin and/or the structural analog(s) and/or compositions of the present disclosure. In various examples, a method of downregulating of UGT1A10, at least partially eliminating TRA-1-60 and/or TRA-1-81 expression, or increasing chemosensitivity of chemoresistant cancer cells (CCCs), if present, or any combination thereof in a subject comprises: administering to a subject an effective amount of Cymarin and/or a structural analog or analogs thereof and/or one or more pharmaceutically acceptable salt, a salt, a partial salt, a solvate, a hydrate, a polymorph, or a prodrug, or the like of the Cymarin and/or the structural analog(s) and/or a stereoisomer or a mixture of stereoisomers, an isotopic variant, or a tautomer, or the like of the Cymarin and/or the structural analog(s) and/or one or more composition(s) (e.g., pharmaceutical composition(s)) of the present disclosure, where UGT1A10 is downregulated, TRA-1-60 and/or TRA-1-81 expression is at least partially eliminated or chemosensitivity of chemoresistant cancer cells (CCCs), if present, is increased in the subject. In various examples, TRA-1-60 and/or TRA-1-81 expression is completely eliminated.

In various examples, a method for treating one or more cancer(s) and/or one or more chemoresistant cancer(s) characterized by TRA-1-60 and TRA-1-81 enrichment in chemosensitivity of chemoresistant cancer cells (CCCs), if present, or a presence of TRA-1-60⁺/TRA-1-81⁺ cells, or any combination thereof, or any combination thereof in a subject comprises administering to subject diagnosed with or in need to treatment for the cancer(s) and/or the chemoresistant cancer(s) an effective amount of Cymarin and/or a structural analog or analogs thereof and/or one or more pharmaceutically acceptable salt, a salt, a partial salt, a solvate, a hydrate, a polymorph, or a prodrug, or the like of the Cymarin and/or the structural analog(s) and/or a stereoisomer or a mixture of stereoisomers, an isotopic variant, or a tautomer, or the like of the Cymarin and/or the structural analog(s) and/or one or more composition(s) (e.g., pharmaceutical composition(s)) of the present disclosure that results in downregulation of UGT1A10, at least partially eliminating TRA-1-60 and/or TRA-1-81 expression or the like, increasing chemosensitivity of chemoresistant cancer cells (CCCs), if present, or the like, or a combination thereof. In various examples, TRA-1-60 and/or TRA-1-81 expression is completely eliminated. In various examples, the cancer is pancreatic cancer, colon cancer, colorectal cancer, lung cancer, breast cancer, or prostate cancer, or the like, or any combination thereof and/or the chemoresistant cancer is chemoresistant pancreatic cancer, chemoresistant colon cancer, chemoresistant colorectal cancer, chemoresistant lung cancer, chemoresistant breast cancer, chemoresistant prostate cancer, or the like, or any combination thereof.

In various examples, a method of treating a subject diagnosed with or is in need of treatment for pancreatic cancer, colon cancer, colorectal cancer, lung cancer, breast cancer, prostate cancer, or any combination thereof comprises administering to a subject an effective amount of Cymarin and/or a structural analog or analogs thereof and/or one or more pharmaceutically acceptable salt, a salt, a partial salt, a solvate, a hydrate, a polymorph, or a prodrug, or the like of the Cymarin and/or the structural analog(s) and/or a stereoisomer or a mixture of stereoisomers, an isotopic variant, or a tautomer, or the like of the Cymarin and/or the structural analog(s) and/or one or more composition(s) (e.g., pharmaceutical composition(s)) of the present disclosure, where one or more symptom(s) and/or indication(s) of the subject is at least partially alleviated. In various examples, the UGT1A10 is downregulated, TRA-1-60 and/or TRA-1-81 expression or the like is at least partially eliminated, or, in the case where one or more of the pancreatic cancer, the colon cancer, colorectal cancer, the lung cancer, the breast cancer, the prostate cancer, or the combination thereof, if present, is a chemoresistant cancer, chemosensitivity of CCCs, if present, is increased in the subject. In various examples, TRA-1-60 and/or TRA-1-81 expression is completely eliminated. In various examples, one or more or all of the cancers is/are a chemoresistant cancer or cancers. In various examples, the pancreatic cancer is chemoresistant pancreatic cancer and/or the colon cancer is chemoresistant colon and/or the colorectal cancer and/or the lung cancer is chemoresistant lung cancer and/or the breast cancer is chemoresistant breast cancer and/or the prostate cancer is chemoresistant prostate cancer. In various examples, one or more symptom(s) and/or indication(s) of the subject is completely alleviated.

### BRIEF DESCRIPTION OF THE FIGURES

For a fuller understanding of the nature and objects of the disclosure, reference should be made to the following detailed description taken in conjunction with the accompanying figures.

FIG. 1 shows TRA-1-60 and TRA-1-81 are enriched in chemoresistant cancer cells. A-Scheme of the antibody-based cell surface marker screen. See also FIG. 8A. B—Flow cytometry plots of TRA-1-60 and TRA-1-81 in the primary screen. See also FIG. 8B. (C, D) Flow cytometry plots (C) and quantification (D) from co-staining of TRA-1-60 and TRA-1-81 antibodies in SW1990 (upper) and SW1990GR (lower) cells. (E, F) Flow cytometry plots (E) and quantification (F) from co-staining with TRA-1-60 and TRA-1-81 antibodies in multiple GR pancreatic cancer cell lines (lower) and their corresponding parental cell lines (upper). (G) Percentage of cells in the TRA-1-81⁺ population in SW1990 cells after acute GEM treatment. See also FIG. 8C. (H) Percentage of cells in the TRA-1-81⁺ population in a panel of cancer cell lines after acute GEM treatment. See also FIG. 8D. (I) Percentage of TRA-1-81⁺ population in SW1990 cell line after acute treatment of PMX or CPT-11. See also FIG. 8E (upper). (J) Percentage of TRA-1-81⁺ population in the A549 cell line after acute treatment of PMX or CPT-11. See also FIG. 8E (lower). (K, L) Immunohistochemistry staining (K) and quantification (L) of TRA-1-60 in control and GEM-resistant SW1990 xenograft tumors. Values in graphs are represented as Mean±SEM. N=3 independent biological replicates. Scale bar, 50 μm (μm=micrometer(s)). p values by unpaired one-tailed student t-test were *p<0.05, **p<0.01.

FIG. 2 shows TRA-1-60⁺/TRA-1-81⁺ cells show higher resistance to gemcitabine treatment. (A) Viability of TRA-1-60⁺/TRA-1-81⁺ cells (black bars) and TRA-1-60/TRA-1-81⁻ cells (white bars) isolated from SW1990 cells cultured in the absence or presence of GEM. (B) Viability of TRA-1-81⁺ and TRA-1-81⁻ cells isolated from A549 cells cultured in the presence of GEM. (C, D) Colony-formation capacity of TRA-1-81⁺ and TRA-1-81⁻ population isolated from (C) SW1990 cells or (D) A549 cells cultured in the absence or presence of GEM. See also FIG. 9B, C. (E-H) The number of spheres derived from TRA-1-81⁺ and TRA-1-81⁻ cells isolated from SW1990 cells (E) or A549 cells (G) cultured in the absence or presence of GEM. See also FIG. 9D, E. Sphere survival rate of SW1990 cells (F) or A549 cells (H) was calculated by dividing the sphere number in GEM treated condition by that of control condition. (I, J) Flow cytometry analysis and quantification of Annexin V⁺ cells in GEM treated (I) SW1990 cells or (J) A549 cells. Finally, the early apoptotic rate in TRA-1-81^{high} population is lower than that in TRA-1-81^{middle} population. FIG. 9F. Values in graphs are represented as Mean±SEM. N=3 independent biological replicates. p values by unpaired one-tailed student t-test were *p<0.05, **p<0.01.

FIG. 3 shows gene expression profiling identifies UGT1A10 as a functional mediator of TRA-1-60 and/or TRA-1-81 expression and chemoresistance. (A) Heat-map from gene expression profiling of SW1990 vs. SW1990GR cells and TRA-1-81⁺ cells vs. TRA-1-81⁻ cells in SW1990 cells. (B) Venn diagram of genes up-regulated in SW1990GR cell line (compared with SW1990 cell line) and TRA-1-81⁺ cells (compared with TRA-1-81⁻ cells). (C, D) Ontology analysis of genes up-regulated in both SW1990GR cells and TRA-1-81⁺ cells with protein properties (C) and function (D). (E) Viability of SW1990GR cells expressing sgRNAs targeting UGT1A10 in the presence of GEM treatment. (F) qRT-PCR analysis of UGT1A10 differential expression in SW1990 vs SW1990GR cells (left), or in TRA-1-81⁺ vs TRA-1-81⁻ SW1990 cells (right). N=3 independent biological replicates for (E, F). p values by unpaired one-tailed student t-test were *p<0.05, **p<0.01.

FIG. 4 shows UGT1A10 enhances both TRA-1-60 and/or TRA-1-81 expression and chemoresistance. (A) Flow cytometry plots of TRA-1-81 expression in UGT-sg SW1990GR cells. (B) Quantification of flow cytometry plots of Annexin V staining in UGT-sg cells. See also FIG. 10A. (C, D) Colony formation capacity (C) and sphere formation capacity (D) of UGT-sg SW1990GR cells. (E) Flow cytometry plots of the TRA-1-81 expression in UGT-OE SW1990 cells. (F-I) Quantification of flow cytometry plots of AnnexinV staining (F), viability (G), colony formation capacity (H), sphere formation capacity (I) of UGTOE-SW1990 cells. The sensitivity to GEM induced cell apoptosis was calculated by dividing the percentage of Annexin V⁺/DAPI− early apoptotic cell population in GEM treated conditions by that of control condition. See also FIG. 10B. Values in graphs are represented as Mean±SEM. N=3 independent biological replicates. p values by unpaired one-tailed student t-test were *p<0.05, **p<0.01.

FIG. 5 shows a high content chemical screen identifies Cymarin as a compound that decreases the percentage of TRA-1-60⁺ cells and increases chemosensitivity. (A) Chemical structure of Cymarin (CYM). See also FIG. 11A, B. (B) Curve for TRA-1-60 inhibition with CYM in SW1990GR cells generated via immunofluorescence. See also FIG. 11C. (C) Immunofluorescence of TRA-1-60 staining in CYM-treated SW1990GR cells. (D, E) Dose curves of CYM on the percentage of TRA-1-81⁺ SW1990GR cells (D) and parental SW1990 cells (E) generated via flow cytometry. See also FIG. 11D. (F, G) Time course of CYM on the percentage of TRA-1-81⁺ SW1990GR cells (F) and parental SW1990 cells (G) generated via flow cytometry. See also FIG. 11E. (H) Quantification of TRA-1-81⁺ SW1990 cells treated with control, 100 nM (nM=nanomolar) GEM, 25 nM CYM or 100 nM GEM plus 25 nM CYM, generated via flow cytometry. See also FIG. 11F (in DU154 and HCT-116 cells) and 5G (in SW1990 cells). (I, J) qRT-PCR analysis of UGT1A10 expression in SW1990GR cells (I) or SW1990 cells (J) with CYM treatment. (K) Viability curves of SW1990 or SW1990GR cell lines with CYM treatment. (L) Growth curves of SW1990 (left) or SW1990GR (right) cell lines with CYM treatment. (M) Viability of SW1990 cells treated with control, 30 nM GEM, 25 nM CYM or 30 nM GEM plus 25 nM CYM. See also FIG. 11I (in HPAF-II and HT-29 cells). Values in graphs are represented as Mean±SEM. N=3 independent biological replicates. Scale bar, 100 m. p values by unpaired one-tailed student t-test were *p<0.05, **p<0.01.

FIG. 6 shows Cymarin blocks tumor growth and increases chemosensitivity in vivo. (A) Representative image of mice transplanted with luciferase labeled-SW1990 cells treated with control, GEM (50 mg/kg bodyweight), CYM (2 mg/kg bodyweight) or GEM (50 mg/kg bodyweight) plus CYM (2 mg/kg bodyweight). (B-D) Growth curves based on luciferase intensity (B), photos (C), weight (D) of tumors treated as in (A). (E, F) immunofluorescent staining (E) and quantification (F) of TRA-1-60⁺ cells in SW1990 xenograft tumors treated as in (A). Scale bar, 50 m. (G, H) immunofluorescent staining (G) and quantification (H) of TUNEL⁺ cells in SW1990 xenograft tumors treated as in (A). Scale bar, 100 m. Values in graphs are represented as Mean±SEM. N=12 independent biological replicates. p values by unpaired one-tailed student t-test were *p<0.05, **p<0.01.

FIG. 7 shows TRA-1-60 specifically stains primary human pancreatic cancer tissues. (A) Representative cores negative (upper) and positive (lower) of TRA-1-60 in commercial TMA. Scale bar: 200 m. (B) Quantitative correlation of TRA-1-60⁺ cores of pancreatic tissue in distinct pathologic conditions. (C) Immunofluorescent staining and (D) quantification of TRA-1-60⁺ cells in patient derived xenograft tumors treated with 50 mg/kg GEM in nude mice. Scale bar: 100 m. (E) Representative cores negative (upper) and positive (lower) of TRA-1-60 in lab-fabricated TMA. Scale bar: 200 m. (F) The number of TRA-1-60⁺ cells, the percentage of TRA-1-60⁺ cells, the total intensity of TRA-1-60⁺ staining, and the total TRA-1-60⁺ area in the positive cores of short and long survival patients. (G) Quantitative correlation of TRA-1-60 status with progression stage of patient tumors. Values in graphs are represented as Mean±SEM. N=3 independent biological replicates. p values by unpaired two-way ANOVA were *p<0.05, **p<0.01.

FIG. 8 shows TRA-1-60 and TRA-1-81 are enriched in chemoresistant cancer cells. See FIG. 1. (A) Viability curve of SW1990 and SW1990GR cells in presence of GEM. (B) Results of primary cell surface marker antibody library screening. X axis presents the absolute difference value, which is calculated by subtracting the percentage of positive cells in SW1990 cells from that of SW1990GR cells. Y axis presents the fold change, which is calculated by dividing the percentage of positive cells in SW1990GR cells by that of SW1990 cells. (C-E) Flow cytometry plots of TRA-1-81 expression acute drug treatment. (C) Flow cytometry plots of TRA-1-81 expression in SW1990 cells treated with GEM, which are quantified in FIG. 1G, (D) Flow cytometry plots of TRA-1-81 in multiple cancer cell lines treated with GEM, which are quantified in FIG. 1H, (E) Flow cytometry plots of TRA-1-81 in SW1990 (upper) and A549 (lower) cell lines treated with PMX or CPT-11, which are quantified in FIG. 1I, J. Values in graphs are represented as Mean of 3 independent biological replicates.

FIG. 9 shows images of colony formation and sphere formation. See FIG. 2. (A) Quantification of average expression intensity of TRA-1-81 in TRA-1-81⁺ SW1990 and TRA-1-81⁺ SW1990GR cells. (B) Phase contrast images of colonies, which is quantified in FIG. 2C. (C) Phase contrast images of colonies, which is quantified in FIG. 2D. (D) Phase contrast images of spheres, which is quantified in FIG. 2E. (E) Phase contrast images of spheres, which is quantified in FIG. 2G. Scale bar, 500 m. (F) Quantification of early apoptosis rate in TRA-1-81 negative, TRA-1-81⁺ middle and TRA-1-81⁺ high in SW1990 cells treated with GEM.

FIG. 10 shows flow cytometry analysis of apoptosis in GEM treated UGT-sg cells or UGT-OE cells. See FIG. 4. (A) Flow cytometry analysis of cell apoptosis of UGT-sg cells, which is quantified in FIG. 4B. (B) Flow cytometry analysis of cell apoptosis of UGT-OE cells, which is quantified in FIG. 4F. Values in graphs are represented as Mean of 3 independent biological replicates.

FIG. 11 shows primary chemical screening results and effects of CYM. See FIG. 5. (A) Scatter plot of primary chemical screening results. (B) Chemical structure of Ouabain. (C) Inhibitory curve of Ouabain. (D) Flow cytometry plots of TRA-1-81 expression of cells treated with different doses of CYM. Upper panel was quantified in FIG. 5D and lower panel in FIG. 5E. (E) Flow cytometry plots of TRA-1-81 expression in cells with different days of 25 nM CYM treatment. Upper panel was quantified in FIG. 5F and lower panel in FIG. 5G. (F) The percentage of TRA-1-81⁺ population in other cancer cell lines, such as DU145 (left) and HCT116 (right), under 25 nM CYM treatment for 5 days. (G) Flow cytometry plots of TRA-1-81 expression of cells treated with 100 nM GEM, 25 nM CYM or 100 nM GEM plus 25 nM CYM. Quantified in FIG. 5H. (H) Flow cytometry plots of Annexin V staining in SW1990GR cells after CYM treatment. (I) Viability of cancer cell lines HPAF-II (left) and HT-29 (right) treated with 50 nM GEM and 25 nM CYM for 5 days. (J) qRT-PCR analysis of UGT1A10 expression in TRA-1-81⁻ and TRA-1-81⁺ SW1990GR cells after 25 nM CYM treatment. (K) Cell viability of control and UGT1A10 overexpressing SW1990 cells treated with 50 nM CYM. Values in graphs are represented as Mean±SEM of 3 independent biological replicates. p values by unpaired one-tailed student t-test were **p<0.01; ***p<0.001.

FIG. 12 shows representative image and summary intensity of mice transplanted with luciferase labeled-SW1990 cells treated with control, GEM (50 mg/kg bodyweight), CYM (2 mg/kg bodyweight) or GEM (50 mg/kg bodyweight) (mg=milligram(s)) plus CYM (2 mg/kg bodyweight). See FIG. 6.

### DETAILED DESCRIPTION OF THE DISCLOSURE

Although claimed subject matter will be described in terms of certain examples, other examples, including examples that do not provide all of the benefits and features set forth herein, are also within the scope of this disclosure. Various structural, logical, and process step changes may be made without departing from the scope of the disclosure.

As used herein, unless otherwise indicated, “about”, “substantially”, or “the like”, when used in connection with a measurable variable (such as, for example, a parameter, an amount, a temporal duration, or the like) or a list of alternatives, is meant to encompass variations of and from the specified value including, but not limited to, those within experimental error (which can be determined by, e.g., a given data set, an art accepted standard, etc. and/or with, e.g., a given confidence interval (e.g. 90%, 95%, or more confidence interval from the mean), such as, for example, variations of +/−10% or less, +1-5% or less, +/−1% or less, and +/−0.1% or less of and from the specified value) or to encompass alternatives to the members of the list that would be recognized by one of ordinary skill in the art as alternatives, where the members and the alternatives may define a genus or sub-genus, insofar such variations in a variable and/or variations in the alternatives are appropriate to perform in the instant disclosure. As used herein, the term “about” may mean that the amount or value in question is the exact value or a value that provides equivalent results or effects as recited in the claims or taught herein. That is, it is understood that amounts, sizes, compositions, parameters, and other quantities and characteristics are not and need not be exact, but may be approximate and/or larger or smaller, as desired, reflecting tolerances, conversion factors, rounding off, measurement error, or the like, or other factors known to those of skill in the art such that equivalent results or effects are obtained. In general, an amount, size, composition, parameter, or other quantity or characteristic, or alternative is “about” or “the like,” whether or not expressly stated to be such. It is understood that where “about,” is used before a quantitative value, the parameter also includes the specific quantitative value itself, unless specifically stated otherwise.

Ranges of values are disclosed herein. The ranges set out a lower limit value and an upper limit value. Unless otherwise stated, the ranges include the lower limit value, the upper limit value, and all values between the lower limit value and the upper limit value, including, but not limited to, all values to the magnitude of the smallest value (either the lower limit value or the upper limit value) of a range. It is to be understood that such a range format is used for convenience and brevity, and thus, should be interpreted in a flexible manner to include not only the numerical values explicitly recited as the limits of the range, but also to include all the individual numerical values or sub-ranges encompassed within that range as if each numerical value and sub-range is explicitly recited. To illustrate, a numerical range of “0.1% to 5%” should be interpreted to include not only the explicitly recited values of 0.1% to 5%, but also, unless otherwise stated, include individual values (e.g., 1%, 2%, 3%, and 4%) and the sub-ranges (e.g., 0.5% to 1.1%; 0.5% to 2.4%; 0.5% to 3.2%, and 0.5% to 4.4%, and other possible sub-ranges) within the indicated range. It is also understood (as presented above) that there are a number of values disclosed herein, and that each value is also herein disclosed as “about” that particular value in addition to the value itself. For example, if the value “10” is disclosed, then “about 10” is also disclosed. Ranges can be expressed herein as from “about” one particular value, and/or to “about” another particular value. Similarly, when values are expressed as approximations, by use of the antecedent “about, it will be understood that the particular value forms a further disclosure. For example, if the value “about 10” is disclosed, then “10” is also disclosed.

As used herein, unless otherwise stated, the term “group” refers to a chemical entity that is monovalent (i.e., has one terminus that can be covalently bonded to other chemical species), divalent, or polyvalent (i.e., has two or more termini that can be covalently bonded to other chemical species). The term “group” also includes radicals (e.g., monovalent and multivalent, such as, for example, divalent radicals, trivalent radicals, and the like). Illustrative, non-limiting examples of groups include:

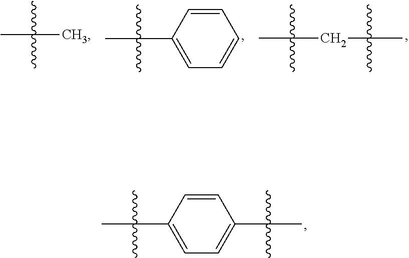

and the like.

As used herein, unless otherwise stated, the term “structural analog” refers to any compound that can be envisioned to arise from an original compound (e.g., Cymarin or the like) if one atom or group of atoms, functional groups, or substructures is replaced with another atom or group of atoms, functional groups, substructures, or the like. In various examples, the term “structural analog” refers to any group that is derived from an original compound ((e.g., Cymarin or the like) by a chemical reaction, where the compound is modified or partially substituted such that at least one structural feature of the compound or group is retained.

The present disclosure provides, inter alia, compositions. The present disclosure also provides methods, such as, for example, methods of treating cancer.

In an aspect, the present disclosure provides compositions. In various examples, a composition is a pharmaceutical composition. Non-limiting examples of the compositions are described herein.

In various examples, a composition (such as, for example, a pharmaceutical composition) comprises (or consists essentially of) Cymarin (also referred to as Cymarine or CYM) and/or one or more Cymarin structural analog(s) and/or one or more pharmaceutically acceptable salt, a salt, a partial salt, a solvate, a hydrate, a polymorph, a prodrug, or the like thereof and/or a stereoisomer or a mixture of stereoisomers, an isotopic variant, a tautomer, or the like thereof. In various examples, a composition comprises (or consists essentially of) one or more compound(s) independently comprising the following structure:

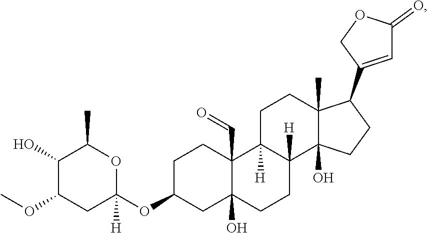

or a structural analog thereof, and/or a pharmaceutically acceptable salt, a salt, a partial salt, a solvate, a hydrate, a polymorph, a prodrug, or the like thereof, and/or a stereoisomer or a mixture of stereoisomers, an isotopic variant, a tautomer, or the like thereof. In various examples, a structural analog of Cymarin exhibits similar activity or substantially similar activity to Cymarin in one or more method(s) of the present disclosure. Non-limiting and illustrative examples of structural analogs of Cymarin include Ouabain comprising the following structure:

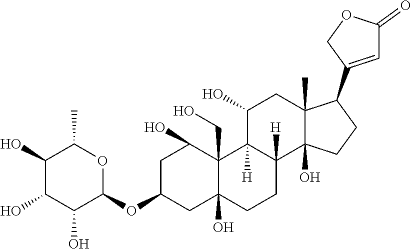

and pharmaceutically acceptable salts, a salt, a partial salt, a solvate, a hydrate, a polymorph, a prodrug, or the like thereof, and a stereoisomer or a mixture of stereoisomers, an isotopic variant, a tautomer, or the like thereof. In various examples, a composition comprises Ouabain comprising the following structure:

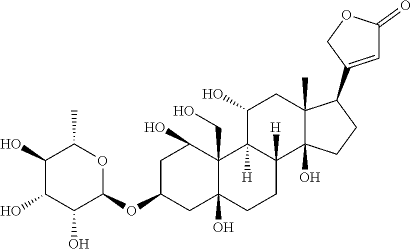

or a structural analog thereof, and/or a pharmaceutically acceptable salt, a salt, a partial salt, a solvate, a hydrate, a polymorph, a prodrug, or the like thereof, and/or a stereoisomer or a mixture of stereoisomers, an isotopic variant, a tautomer, or the like thereof. In various examples, a composition further comprises one or more additional component(s), one or more or all of which may be pharmaceutically acceptable components. Non-limiting examples of compositions are described herein.

As used herein, unless otherwise indicated, the term “pharmaceutically acceptable” refers to those components and dosage forms that are, within the scope of sound medical judgment, suitable for use in contact with the tissues of humans or animals without undesirable or excessive toxicity, irritation, or other problem or complication, which may be commensurate with a reasonable benefit/risk ratio.

Some non-limiting examples of materials which can be used as additional component(s) in a composition include sugars, such as, for example, lactose, glucose, sucrose, and the like; starches, such as, for example, corn starch, potato starch, and the like; cellulose, and its derivatives, such as, for example, sodium carboxymethyl cellulose, ethyl cellulose, cellulose acetate, and the like; powdered tragacanth; malt; gelatin; talc; excipients, such as, for example, cocoa butter, suppository waxes, and the like; oils, such as, for example, peanut oil, cottonseed oil, safflower oil, sesame oil, olive oil, corn oil, soybean oil, and the like; glycols, such as, for example, propylene glycol and the like; polyols, such as, for example, glycerin, sorbitol, mannitol, polyethylene glycol, and the like; esters, such as, for example, ethyl oleate, ethyl laurate, and the like; agar; buffering agents, such as, for example, magnesium hydroxide, aluminum hydroxide, and the like; alginic acid; pyrogen-free water; isotonic saline; Ringer's solution; ethyl alcohol; phosphate buffer solutions; and other non-toxic compatible substances employed in pharmaceutical formulations. (See, e.g., REMINGTON'S PHARM. SCI., 15th Ed. (Mack Publ. Co., Easton (1975)). In various examples, an additional component is a pharmaceutically acceptable carrier. Non-limiting examples of pharmaceutically acceptable carriers are found in: Remington: The Science and Practice of Pharmacy (2012) 22nd Edition, Philadelphia, PA. Lippincott Williams & Wilkins.

In an aspect, the present disclosure provides uses of Cymarin and/or a structural analog or analogs thereof and/or composition(s) of the present disclosure. “Cymarin and/or a structural analog or analogs thereof,” as used in the methods herein, unless otherwise stated, includes and/or one or more pharmaceutically acceptable salt, a salt, a partial salt, a solvate, a hydrate, a polymorph, a prodrug, or the like of the Cymarin and/or structural analogs and/or a stereoisomer or a mixture of stereoisomers, an isotopic variant, a tautomer, or the like of the Cymarin and/or structural analogs.

In various examples, Cymarin and/or a structural analog or analogs thereof and/or composition(s) are used to treat a cancer or a combination of cancers (any one of which or all of which are a chemoresistant cancer or the like). Non-limiting examples of uses of Cymarin and/or a structural analog or analogs thereof and/or composition(s) of the present disclosure are described herein.

In various examples, Cymarin and/or a structural analog or analogs thereof and/or composition(s) of the present disclosure is/are used in treatment methods. In various examples, a method of treatment comprises (or consists essentially of or consists of) administration of Cymarin and/or a structural analog or analogs thereof and/or composition(s) of the present disclosure to a subject. In various examples, a subject comprises TRA-1-60⁺ and/or TRA-1-81⁺ cells or the like. In various examples, a method treats a cancer or cancers (such as, for example, pancreatic cancer, colon cancer, colorectal cancer, lung cancer, breast cancer, prostate cancer, chemoresistant pancreatic cancer, chemoresistant colon cancer, chemoresistant colorectal cancer, chemoresistant lung cancer, chemoresistant breast cancer, chemoresistant prostate cancer, or the like, or any combination thereof). In various examples, one or more of the cancer(s) is/are a chemoresistant cancer or cancers. In various examples, a method targets pancreatic cancer cells, colon cancer cells, colorectal cancer cells, lung cancer cells, breast cancer cells, prostate cancer cells or the like, or any combination thereof, any of which or any portion of which may independently be cancer stem cells or the like. In various examples, a method targets chemoresistant pancreatic cancer cells, chemoresistant colon cancer cells, chemoresistant colorectal cancer cells, chemoresistant lung cancer cells, chemoresistant breast cancer cells, chemoresistant prostate cancer cells or the like, or any combination thereof or the like, any of which may independently be chemoresistant cancer stem cells or the like.

In various examples, a method results in downregulation of UGT1A10 or the like, at least partially or completely eliminates TRA-1-60 and/or TRA-1-81 expression or the like, increases chemosensitivity of chemoresistant cancer cells (CCCs) (such as, for example, chemoresistant pancreatic cancer cells, chemoresistant colon cancer cells, chemoresistant colorectal cancer cells, chemoresistant lung cancer cells, chemoresistant breast cancer cells, chemoresistant prostate cancer cells or the like, or any combination thereof) or the like, any of which may independently be chemoresistant cancer stem cells or the like, if present, or the like, or any combination thereof, in vitro or in vivo (e.g., in a subject). In various examples, a method of downregulating of UGT1A10 or the like, at least partially or completely eliminating TRA-1-60 and/or TRA-1-81 expression or the like, or increasing chemosensitivity of chemoresistant cancer cells (CCCs) (such as, for example, chemoresistant pancreatic cancer cells, chemoresistant colon cancer cells, chemoresistant colorectal cancer cells, chemoresistant lung cancer cells, chemoresistant breast cancer cells, chemoresistant prostate cancer cells or the like, or any combination thereof) or the like, any of which may independently be chemoresistant cancer stem cells or the like, if present, or the like, or any combination thereof in a subject, comprises administering to a subject an amount of Cymarin and/or a structural analog or analogs thereof and/or composition(s) (which may be an effective amount), where UGT1A10 or the like is downregulated, TRA-1-60 and/or TRA-1-81 expression or the like is at least partially or completely eliminated, chemosensitivity of chemoresistant cancer cells (CCCs) (such as, for example, chemoresistant pancreatic cancer cells, chemoresistant colon cancer cells, chemoresistant colorectal cancer cells, chemoresistant lung cancer cells, chemoresistant breast cancer cells, chemoresistant prostate cancer cells or the like, or any combination thereof), or the like, any of which may independently be chemoresistant cancer stem cells or the like, if present, is increased, or the like, in the subject.

In various examples, the present disclosure provides a means for downregulating of UGT1A10 or the like, at least partially or completely eliminating TRA-1-60 and/or TRA-1-81 expression or the like, or increasing chemosensitivity of chemoresistant cancer cells (CCCs) (such as, for example, chemoresistant pancreatic cancer cells, chemoresistant colon cancer cells, chemoresistant colorectal cancer cells, chemoresistant lung cancer cells, chemoresistant breast cancer cells, chemoresistant prostate cancer cells or the like, or any combination thereof) or the like, any of which may independently be chemoresistant cancer stem cells or the like, if present, or the like, or any combination thereof in vitro or in vivo (e.g., in a subject).

In various examples, a method of downregulating of UGT1A10 or the like, at least partially or completely eliminating TRA-1-60 and/or TRA-1-81 expression or the like, or increasing chemosensitivity of chemoresistant cancer cells (CCCs) (such as, for example, chemoresistant pancreatic cancer cells, chemoresistant colon cancer cells, chemoresistant colorectal cancer cells, chemoresistant lung cancer cells, chemoresistant breast cancer cells, chemoresistant prostate cancer cells or the like, or any combination thereof) or the like, any of which may independently be chemoresistant cancer stem cells or the like, if present, or the like, or any combination thereof in a subject, comprises administering to a subject an amount of Cymarin and/or a structural analog or analogs thereof and/or composition(s) (which may be an effective amount), where UGT1A10 or the like is downregulated, TRA-1-60 and/or TRA-1-81 expression or the like is at least partially, substantially, or completely eliminated, chemosensitivity of chemoresistant cancer cells (CCCs) (such as, for example, chemoresistant pancreatic cancer cells, chemoresistant colon cancer cells, chemoresistant colorectal cancer cells, chemoresistant lung cancer cells, chemoresistant breast cancer cells, chemoresistant prostate cancer cells or the like, or any combination thereof) or the like, any of which may independently be chemoresistant cancer stem cells or the like, if present, is increased, or the like, in the subject.

In various examples, downregulation of UGT1A10 or the like is compared to a subject that has not been administered Cymarin and/or a structural analog or analogs thereof and/or composition(s), at least partial, substantial elimination, or completely elimination of TRA-1-60 and/or TRA-1-81 expression or the like is compared to a subject that has not been administered Cymarin and/or a structural analog or analogs thereof and/or composition(s) and/or increasing chemosensitivity of chemoresistant cancer cells (CCCs) (such as, for example, chemoresistant pancreatic cancer cells, chemoresistant colon cancer cells, chemoresistant colorectal cancer cells, chemoresistant lung cancer cells, chemoresistant breast cancer cells, chemoresistant prostate cancer cells or the like, or any combination thereof) or the like, any of which may independently be chemoresistant cancer stem cells or the like, if present, is compared to a subject (or the subject) that has not been administered Cymarin and/or a structural analog or analogs thereof and/or composition(s), which may be determined in vitro or in vivo or the like.

In various examples, downregulation of UGTIA10 or the like is at least about at least about 20% or greater, at least about 25% or greater, at least about 30% or greater, at least about 40% or greater, at least about 50% or greater, at least about 60% or greater (any of the foregoing ranges may have an upper bound of about 100%) (e.g., compared to the subject prior to administration of Cymarin and/or a structural analog or analogs thereof and/or composition(s)), and/or TRA-1-60 and/or TRA-1-81 expression is at least about 20% or greater, at least about 25% or greater, at least about 30% or greater, at least about 40% or greater, at least about 50% or greater, at least about 60% or greater (any of the foregoing ranges may have an upper bound of about 100%) (e.g., compared to the subject prior to administration of Cymarin and/or a structural analog or analogs thereof and/or composition(s)) and/or increasing chemosensitivity of chemoresistant cancer cells (CCCs) (such as, for example, chemoresistant pancreatic cancer cells, chemoresistant colon cancer cells, chemoresistant colorectal cancer cells, chemoresistant lung cancer cells, chemoresistant breast cancer cells, chemoresistant prostate cancer cells or the like, or any combination thereof), or the like, any of which may independently be chemoresistant cancer stem cells, if present, is effectiveness (e.g., observable or demonstrable effectiveness, which may be determined by a method or methods known in the art) or increased effectiveness (e.g., observable or demonstrable increase in effectiveness of at least at least about 20% or greater, at least about 25% or greater, at least about 30% or greater, at least about 40% or greater, at least about 50% or greater, at least about 60% or greater (any of the foregoing ranges may have an upper bound of about 100%) (e.g., compared to the subject prior to administration of Cymarin and/or a structural analog or analogs thereof and/or composition(s)) of other chemotherapeutic agent(s)). In various examples, a chemotherapeutic agent that was not effective in treating a subject for cancer (such as, for example, pancreatic cancer (e.g., chemoresistant pancreatic cancer or the like) or the like) is effective in treating the subject after administration of Cymarin and/or a structural analog or analogs thereof and/or composition(s).

In various examples, a method treats a subject diagnosed with or in need of treatment for cancer (such as for example, pancreatic cancer, colon cancer, colorectal cancer, lung cancer, breast cancer, prostate cancer, or the like, or any combination thereof, any of which may independently be a chemoresistant cancer or the like. In various examples, a method for treating a subject diagnosed with or in need of treatment for cancer (such as, for example, pancreatic cancer, colon cancer, colorectal cancer, lung cancer, breast cancer, prostate cancer, or the like, or any combination thereof, any of which may independently be a chemoresistant cancer or the like) comprises administering to a subject an amount (which may be an effective amount) of Cymarin and/or a structural analog or analogs thereof and/or composition(s), where one or more symptom(s), indication(s), or the like, or any combination thereof, of the subject is at least partially alleviated. In various examples, at least one of the cancer(s) is pancreatic cancer and the pancreatic cancer is pancreatic cancer sub-type, such as, for example, classical-like pancreatic cancer, basal-like pancreatic cancer, or the like.

In various examples, a method treats pancreatic cancer, colon cancer, colorectal cancer, lung cancer, breast cancer, prostate cancer, or the like, or any combination thereof, any of which may independently be a chemoresistant cancer or the like, characterized by TRA-1-60 and/or TRA-1-81, two “sternness” cell surface markers, enrichment in CCCs or the like, or TRA-1-60⁺/TRA-1-81⁺ cells or the like, or the like, or any combination thereof. In various examples, at least one of the cancer(s) is pancreatic cancer and the pancreatic cancer is a pancreatic cancer sub-type, such as, for example, classical-like pancreatic cancer, basal-like pancreatic cancer, or the like.

In various examples, treatment of subject with Cymarin and/or a structural analog or analogs thereof and/or composition(s) potentiates a chemotherapeutic agent treatment. In various examples, a exhibits a synergistic anti-cancer response.

In various examples, a method further comprises administration of one or more chemotherapeutic agent(s) (which are not Cymarin or a structural analog thereof). Examples of suitable chemotherapeutic agents are known in the art. In various examples, a chemotherapeutic agent is a chemotherapeutic agent used to treat pancreatic cancer, colon cancer, colorectal cancer, lung cancer, breast cancer, prostate cancer, or the like, or any combination thereof, or the like. Non-limiting examples of chemotherapeutic agents include gemcitabine, 5-fluorouracil (5-FU) drugs, capecitabine, anthracyclines (such as, for example, doxorubicin, epirubicin, and the like), taxanes (such as, for example, paclitaxel (Taxol), Albumin-bound paclitaxel (Abraxane), docetaxel (Taxotere), cabazitaxel, and the like) and FILFIRINOX, mitoxantrone, estramustine, cyclophosphamide (Cytoxan), etoposide, irinotecan, topotecan, cisplatin, carboplatin, oxaliplatin, trifluridine and tipiracil, and the like, and prodrugs thereof, and any combination thereof. Non-limiting examples, of chemotherapeutic agents include those approved for treatment of pancreatic cancer, colon cancer, colorectal cancer, lung cancer, breast cancer, prostate cancer, or the like, or any combination thereof by the U.S. Food and Drug Administration.

In various examples, Cymarin and/or a structural analog or analogs thereof and/or composition(s) is/are administered prior to and/or concurrently administering one or more chemotherapeutic agent(s). In various examples, one or more chemotherapeutic agent(s) is/are administered within about 1 to about 24 hours, including all 0.1 hour values and ranges therebetween, or about 1 to about 3 days, including all 0.1 hour values and ranges therebetween.

In various examples, treatment of a subject (or administration of Cymarin and/or a structural analog or analogs thereof and/or composition(s)) results in a decrease in size (e.g., a decrease in size (e.g., linear dimension, volume, or the like, or any combination thereof) or weight, or both, of at least about 10% or greater, at least about 15% or greater, at least about 20% or greater, at least about 25% or greater, at least about 30% or greater, at least about 40% or greater, at least about 50% or greater, or at least about 60% or greater (any of the foregoing ranges may have an upper bound of about 100%) (e.g., compared to the subject prior to administration of Cymarin and/or a structural analog or analogs thereof and/or composition(s)) of a primary tumor or tumor(s). In various examples, treatment of a subject (or administration of Cymarin and/or a structural analog or analogs thereof and/or composition(s)) inhibits or prevents metathesis of a cancer or cancers (such as, for example, pancreatic cancer, colon cancer, colorectal cancer, lung cancer, breast cancer, prostate cancer, or the like, or any combination thereof, any of which may independently be a chemoresistant cancer or the like). In various examples, treatment of a subject (or administration of Cymarin and/or a structural analog or analogs thereof and/or composition(s)) results in a decrease in size of a primary tumor or tumor(s) and inhibits or prevents metathesis of a cancer (such as, for example, pancreatic cancer, colon cancer, colorectal cancer, lung cancer, breast cancer, prostate cancer, or the like, or any combination thereof, any of which may independently be a chemoresistant cancer or the like). In various examples, treatment of a subject (or administration of Cymarin and/or a structural analog or analogs thereof and/or composition(s)) prevents or inhibits relapse of a cancer (such as, for example, pancreatic cancer, colon cancer, colorectal cancer, lung cancer, breast cancer, prostate cancer, or the like, or any combination thereof, any of which may independently be a chemoresistant cancer or the like).

A subject (e.g., a subject in need of treatment or the like) may be a human or other animal (which may be a non-human mammal). Non-limiting examples of non-human animals (which may be mammals) include cows, pigs, mice, rats, rabbits, cats, dogs, and other agricultural animals, pets (such as, for example, dogs, cats, and the like), service animals, and the like.

In various examples, a subject in need of treatment has been diagnosed with or is suspected of having one or more cancer(s) (such as, for example, pancreatic cancer, colon cancer, colorectal cancer, lung cancer, breast cancer, prostate cancer, or the like, or any combination thereof, any of which may independently be a chemoresistant cancer or the like). In various examples, the cancer is a solid tumor or the like. In various examples, a subject is diagnosed or a prognosis is made for the individual based on expression of described genes as determined from a biological sample from the subject. The biological sample can be analyzed using any suitable technique, such as, for example, immunoassays for proteins, and sequencing for nucleic acid analysis, and the like, and any combination thereof. Results from analyzing the sample can be compared to any suitable control, such as, for example, a value obtained for expression of the same gene or genes from an individual who does not have cancer. In various examples, inhibition of expression of a gene and/or inhibition of production and/or function of a protein encoded by the gene, can be determined by comparison to a suitable control. In various examples, the control is a value obtained for expression of a gene from one or more samples that have not been treated with a described agent.

“Treating” or “treatment” of pancreatic cancer (such as, for example, chemoresistant pancreatic cancer or the like) or the like refers, in various examples, to ameliorating (e.g., arresting, reversing, alleviating, or the like) the pancreatic cancer (such as, for example, chemoresistant pancreatic cancer or the like) or the like, one or more side effect(s) thereof, or the like, or a combination thereof, or reducing the manifestation, extent or severity of one or more clinical symptom(s) or the like of the cancer(s) (such as, for example, pancreatic cancer, colon cancer, colorectal cancer, lung cancer, breast cancer, prostate cancer, or the like, or any combination thereof, any of which may independently be a chemoresistant cancer or the like). In various other examples, “treating” or “treatment” refers to ameliorating one or more physical parameter(s), which, independently, may or may not be discernible by the subject. In yet other examples, “treating” or “treatment” refers to modulating the pancreatic cancer (such as, for example, chemoresistant pancreatic cancer or the like) or the like, one or more side effect(s) thereof, or the like, or a combination thereof, either physically, (e.g., stabilization of one or more discernible symptom(s), or the like), physiologically, (e.g., stabilization of one or more physical parameter, or the like), or both. In yet other examples, treating” or “treatment” relates to slowing the progression of the cancer(s) (such as, for example, pancreatic cancer, colon cancer, colorectal cancer, lung cancer, breast cancer, prostate cancer, or the like, or any combination thereof, any of which may independently be a chemoresistant cancer or the like) or the like, one or more side effect(s) thereof, or the like, or a combination thereof. Treating may include administration of an effective amount of the Cymarin and/or a structural analog or analogs thereof and/or composition(s).

As used herein, unless otherwise indicated, the term “effective amount” means that amount of the Cymarin and/or a structural analog or analogs thereof and/or composition(s) that will elicit the biological or medical response of a subject (or a tissue, system, or the like, thereof) that is being sought, for instance, by a researcher, clinician, or the like. An effective amount may be a therapeutically effective amount. The term “therapeutically effective amount” includes any amount which, as compared to a corresponding subject or the subject who has not received such amount, results in improved treatment, healing, prevention, or amelioration of the pancreatic cancer (such as, for example, chemoresistant pancreatic cancer or the like) or the like, one or more side effect(s) thereof, or the like, or a combination thereof or a decrease in the rate of advancement of a cancer (such as, for example, pancreatic cancer (e.g., chemoresistant pancreatic cancer or the like) or the like) or the like, one or more side effect(s) thereof, or the like, or a combination thereof. The term also includes within its scope amounts effective to enhance normal physiological function.

A physician or veterinarian having ordinary skill in the art can readily determine and prescribe the effective amount of the compound(s) and/or composition(s) required. The selected dosage level can depend upon a variety of factors including, but not limited to, the activity of the particular composition employed, the time of administration, the rate of excretion or metabolism of the particular composition being employed, the rate and extent of absorption, the duration of the treatment, other drugs, compounds and/or materials used in combination with the particular composition employed, the age, sex, weight, condition, general health and prior medical history of the patient being treated, and like factors well known in the medical arts. For example, the physician or veterinarian could start doses of the composition employed at levels lower than that required in order to achieve the desired therapeutic effect and gradually increase the dosage until the desired effect is achieved.

Cymarin and/or a structural analog or analogs thereof and/or composition(s) can be administered by various routes. In various examples, administration comprises a single dose or multiple doses.

Administration can be accomplished by various routes, such as, for example, by parenteral delivery, catheter-based delivery, infusion, injection, cannula delivery, central line delivery, or oral delivery, or the like, or any combination thereof. Non-limiting examples of parenteral delivery include subcutaneous, intra-venous, intra-muscular, intra-arterial, intrathecal, intraperitoneal, and injection into the tissue of an organ, and the like, and any combination thereof. In various examples, catheter-based delivery included delivery by iontophoretic catheter-based delivery or the like. In various examples, oral delivery includes delivery of a pill (which may be an enteric coated pill or the like), or administration of a liquid by mouth, or the like.

A method can be carried out in conjunction with one or more other therapeutic modalit(ies) (e.g., cancer therapeutic modalit(ies) (such as, for example, pancreatic cancer, colon cancer, colorectal cancer, lung cancer, breast cancer, prostate cancer, chemoresistant pancreatic cancer, chemoresistant colon cancer, chemoresistant colorectal cancer, chemoresistant lung cancer, chemoresistant breast cancer, chemoresistant prostate cancer or the like therapeutic modalities) or the like). Examples of suitable other therapeutic modalities are known in the art. In various examples, a method further comprises, one or more radiation treatment(s), one or more photodynamic therapy treatment(s), one or more surgical intervention(s) (e.g., surgical procedure(s), or the like), or the like, or any combination thereof.

In an aspect, the present disclosure provides kits. Non-limiting examples of kits are provided herein.

In various examples, a kit comprises Cymarin and/or a structural analog or analogs thereof and/or composition(s) of the present disclosure. In various examples, a kit includes a closed or sealed package that contains Cymarin and/or a structural analog or analogs thereof and/or composition(s). In various examples, the package comprises one or more closed or sealed vials, bottles, blister (bubble) packs, or any other suitable packaging for the sale, distribution, or use of the Cymarin and/or a structural analog or analogs thereof and/or composition(s). The printed material may include printed information. The printed information may be provided on a label, on a paper insert, printed on a packaging material, or the like. The printed information may include information that identifies the compound(s) and/or composition(s) in the package, the amounts and types of other active and/or inactive ingredients in the composition, and instructions for taking the compound(s) and/or composition(s). The instructions may include information, such as, for example, the number of doses to take over a given period of time, and/or information directed to a pharmacist and/or another health care provider, such as, for example, a physician or the like, or a patient. The printed material may include an indication or indications that the one or more compound(s) and/or composition(s) and/or any other agent provided therein is for treatment of a subject. In various examples, the kit includes a label describing the contents of the kit and providing indications and/or instructions regarding use of the contents of the kit to treat a subject.

The following Statements describe various examples of compositions and of the present disclosure that are not intended to be limiting in any manner:
Statement 1. A composition (such as, for example, a pharmaceutical composition or the like) comprising Cymarin and/or a structural analog or analogs thereof.
Statement 2. A composition (such as, for example, a pharmaceutical composition according to Statement 1, where the pharmaceutical composition further comprises one or more pharmaceutically acceptable excipient(s).
Statement 3. A method of downregulating of UGT1A10 or the like, at least partially or completely eliminating TRA-1-60 and/or TRA-1-81 expression or the like, or increasing chemosensitivity of chemoresistant cancer cells (CCCs) or the like, or the like, or any combination thereof in a subject comprising: administering to a subject an effective amount of Cymarin and/or a structural analog or analogs thereof and/or composition(s) of the present disclosure (e.g., one or more composition(s) of Statement 1 or 2), where UGT1A10 or the like is downregulated, TRA-1-60 and/or TRA-1-81 expression or the like is at least partially or completely eliminated, or chemosensitivity of chemoresistant cancer cells (CCCs) is increased or the like, or the like, or the combination thereof in the subject.
Statement 4. A method for treating a disease state (such as, for example, pancreatic cancer (e.g., chemoresistant pancreatic cancer or the like) or the like) characterized by TRA-1-60 and TRA-1-81 enrichment in CCCs or the like or the presence of TRA-1-60⁺/TRA-1-81⁺ cells or the like, or the like, or any combination thereof comprising: administering to subject diagnosed with or suffering from the disease state an effective amount of one or more compound(s) that results in downregulation of UGT1A10 or the like, at least partially or completely elimination of TRA-1-60 and/or TRA-1-81 expression or the like, increases chemosensitivity of chemoresistant cancer cells (CCCs) or the like, or the like, or the combination thereof (such as, for example, Cymarin and/or a structural analog or analogs thereof and/or composition(s) of the present disclosure (e.g., one or more composition(s) of Statement 1 or 2)).
Statement 5. A method of treating a subject diagnosed with or is in need of treatment for pancreatic cancer (e.g., chemoresistant pancreatic cancer or the like) or the like) comprising: administering to a subject an effective amount of Cymarin and/or a structural analog or analogs thereof and/or composition(s) of the present disclosure (e.g., one or more composition(s) of Statement 1 or 2), where one or more symptom(s) and/or indication(s) of the subject is at least partially or completely.
Statement 6. The method of Statement 5, where the UGT1A10 or the like is downregulated, TRA-1-60 and/or TRA-1-81 expression or the like is at least partially or completely eliminated, or chemosensitivity of chemoresistant cancer cells (CCCs) or the like, or the like, or any combination thereof is increased in the subject.

The steps of the method described in the various examples disclosed herein are sufficient to carry out the methods of the present disclosure. Thus, in an example, a method consists essentially of a combination of the steps of the methods disclosed herein. In another example, a method consists of such steps.

The following example is presented to illustrate the present disclosure. It is not intended to be limiting in any manner.

### Example

This Example describes, inter alia, inorganic imaging agents, compositions, and methods of the present disclosure.

A Targetable Pathway to Eliminate TRA-1-60+/TRA-1-81+Chemoresistant Cancer Cells. Chemoresistance is a primary cause of treatment failure in pancreatic cancer. Identifying cell surface markers specifically expressed in chemoresistant cancer cells (CCCs) could facilitate targeted therapies to overcome chemoresistance. An antibody-based screen was performed and it was found that TRA-1-60 and TRA-1-81, two “sternness” cell surface markers, are highly enriched in CCCs. Furthermore, TRA-1-60⁺/TRA-1-81⁺ cells are chemoresistant compared to TRA-1-60⁻/TRA-1-81⁻ cells. Transcriptome profiling identified UGT1A10, shown to be both necessary and sufficient to maintain TRA-1-60/TRA-1-81 expression and chemoresistance. From a high content chemical screen, Cymarin was identified, which downregulates UGT1A10, eliminates TRA-1-60/TRA-1-81 expression, and increases chemosensitivity both in vitro and in vivo. Finally, TRA-1-60/TRA-1-81 expression is highly specific in primary cancer tissue and positively correlated with chemoresistance and short survival, which highlights their potentiality for targeted therapy. Therefore, a novel CCC surface marker regulated by a pathway that promotes chemoresistance was discovered.

An antibody-based cell surface marker screen that identified TRA-1-60 and TRA-1-81 antibodies recognizing an antigen enriched in chemoresistant pancreatic ductal adenocarcinoma (PDAC) cell lines and primary patient samples is described. TRA-1-60⁺/TRA-1-81⁺ cells are chemoresistant compared to the negative cohort. Global transcriptional profiling identified UGT1A10 as a necessary and sufficient regulator of TRA-1-60/TRA-1-81 expression and chemoresistance. Finally, a high content chemical screen discovered Cymarin, an anti-arrhythmia agent, as a lead drug that decreases UGT1A10 expression, eliminates TRA-1-60⁺/TRA-1-81⁺ cells, and increases chemosensitivity both in vitro and in vivo.

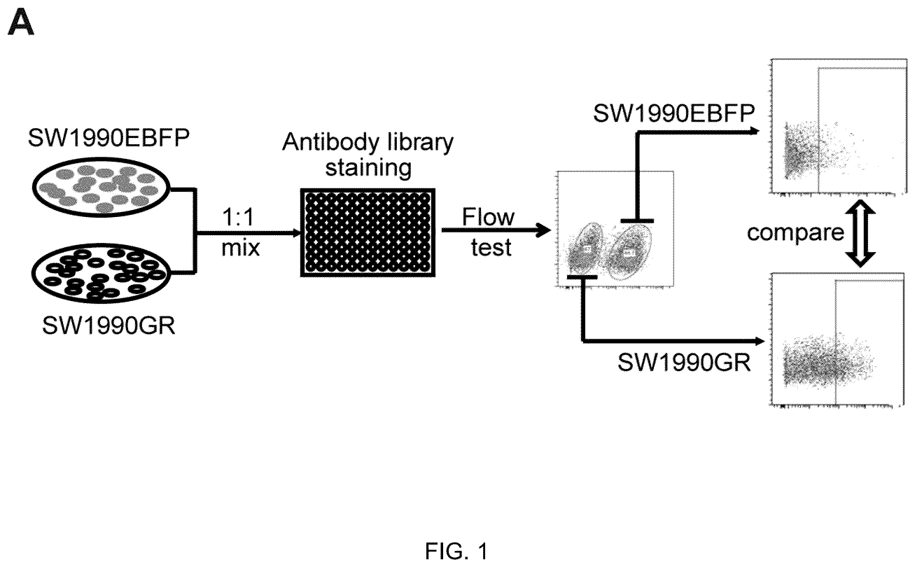

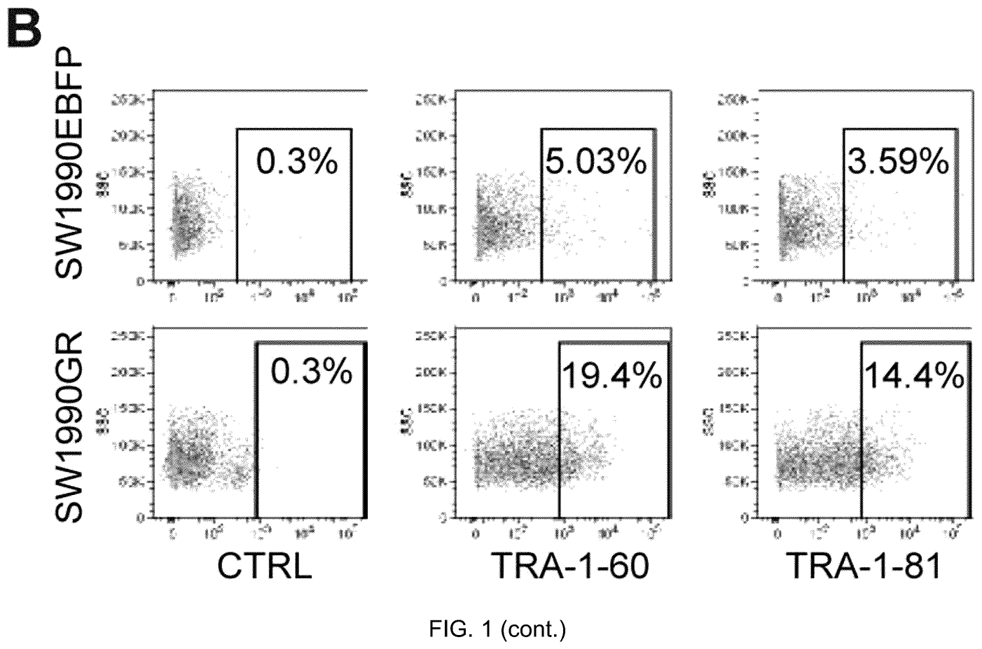

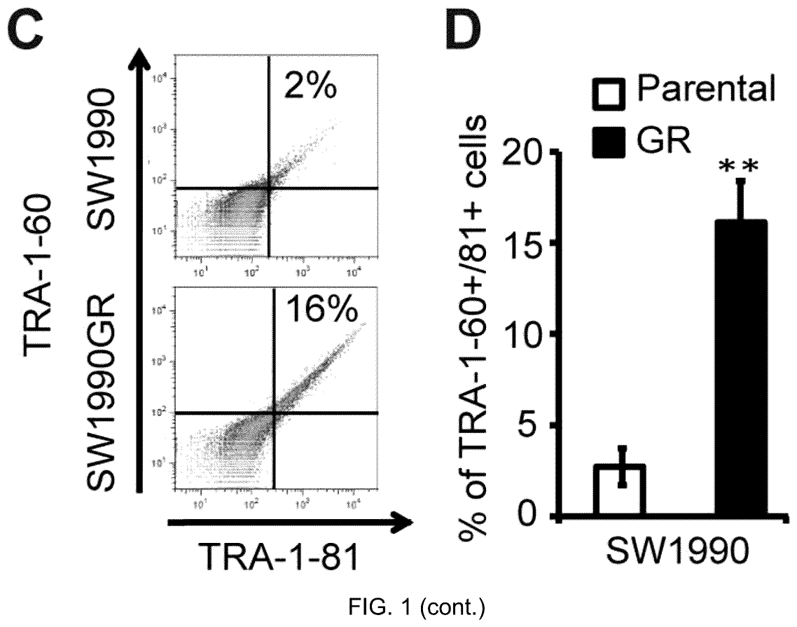

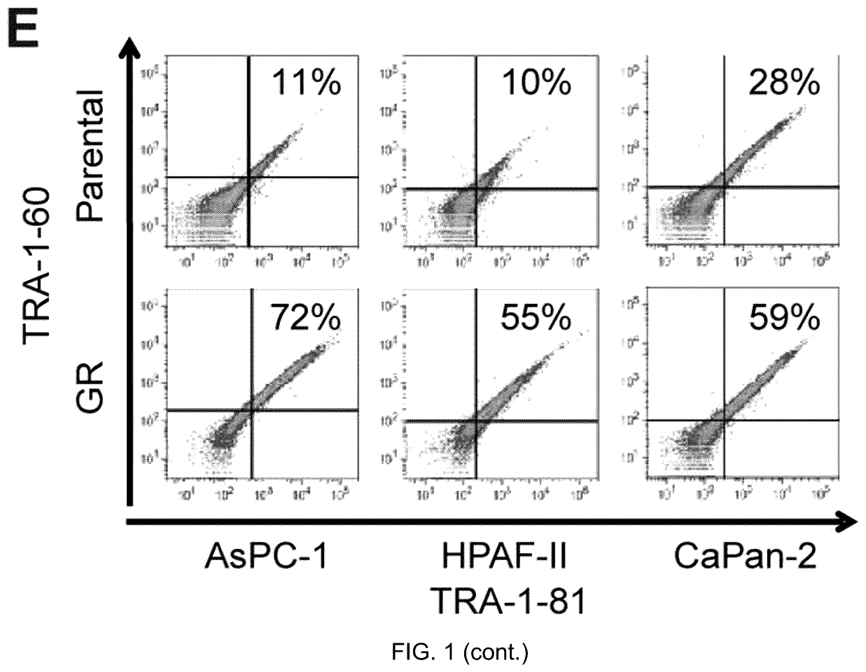

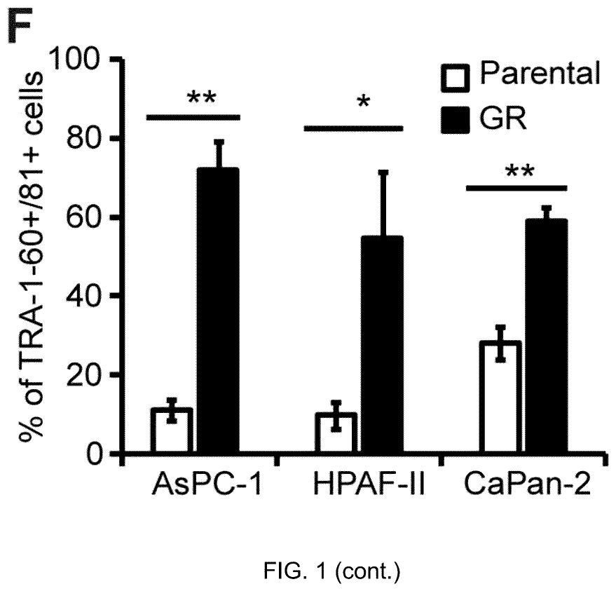

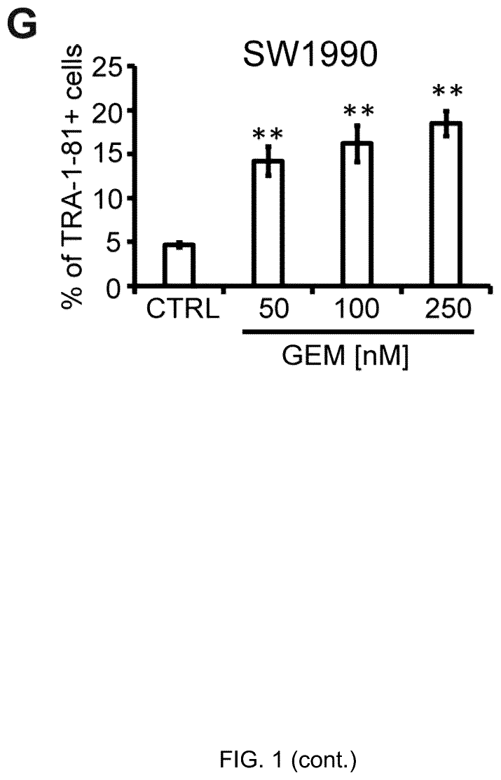

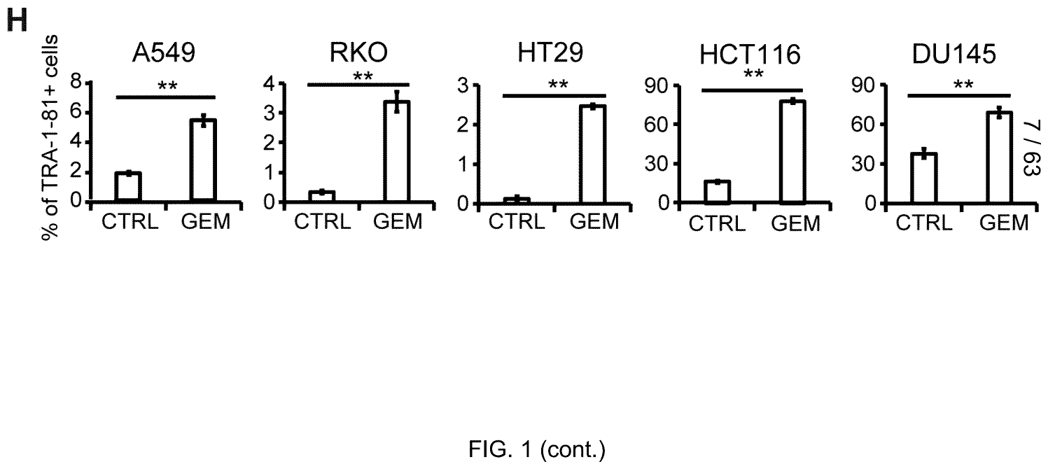

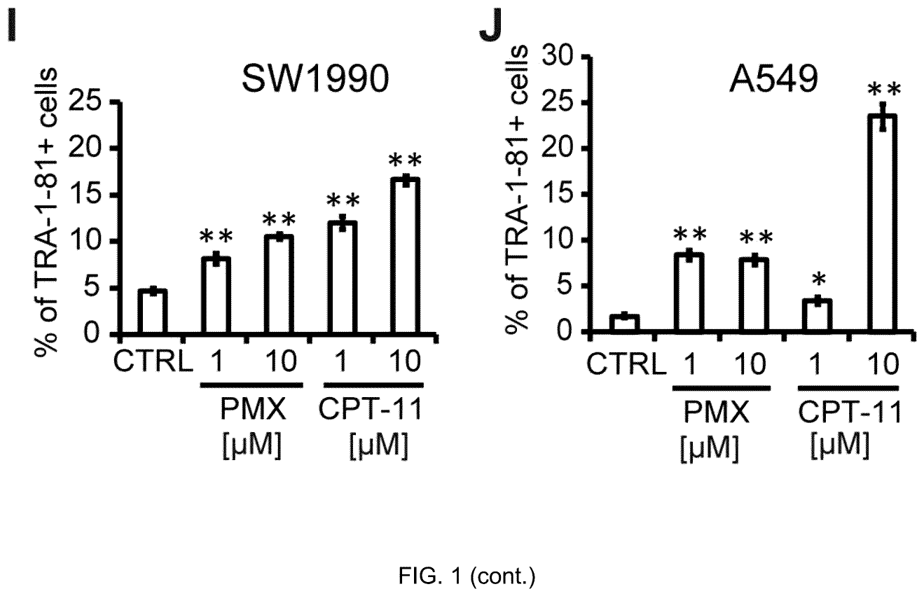

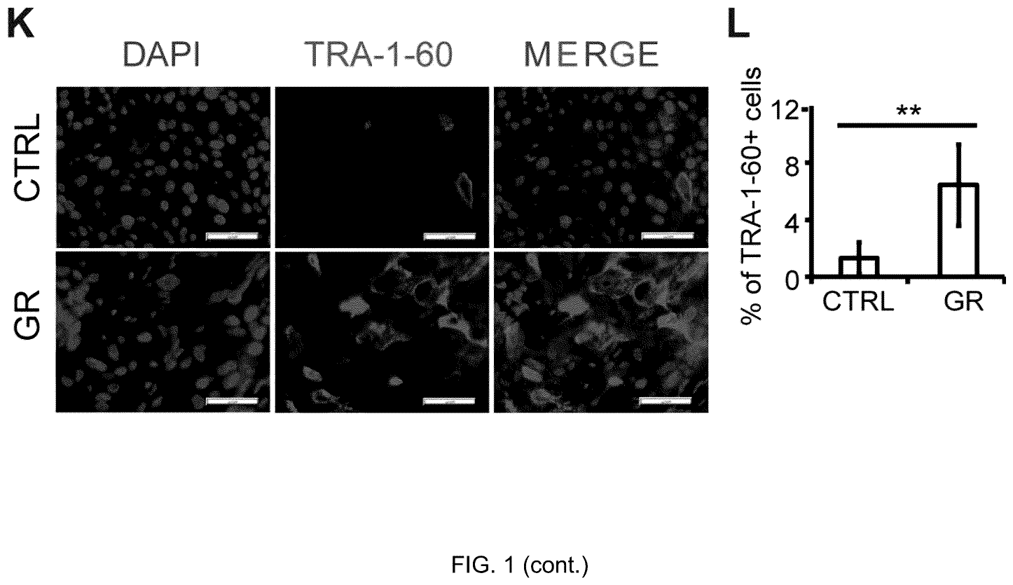

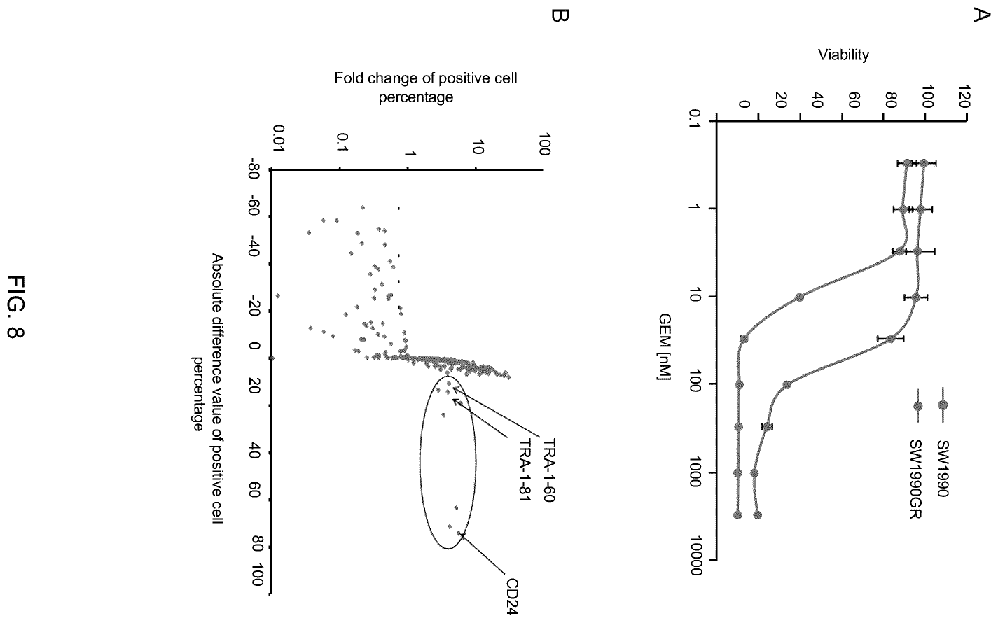

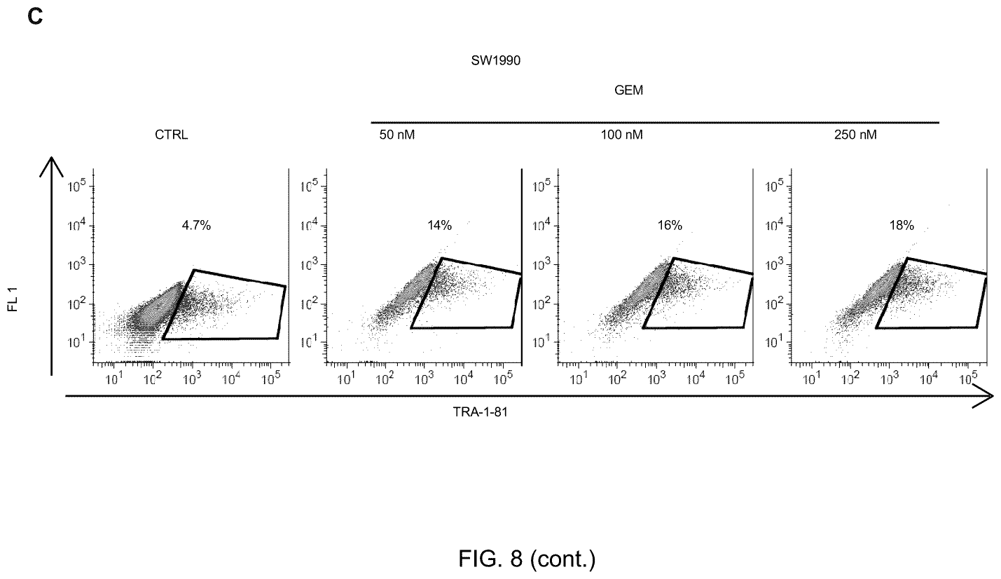

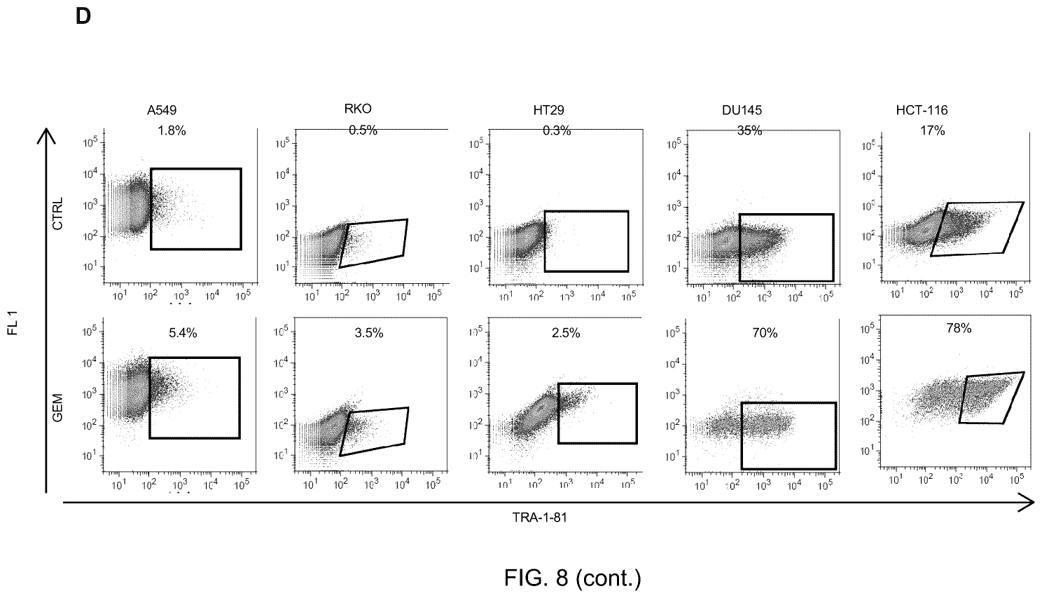

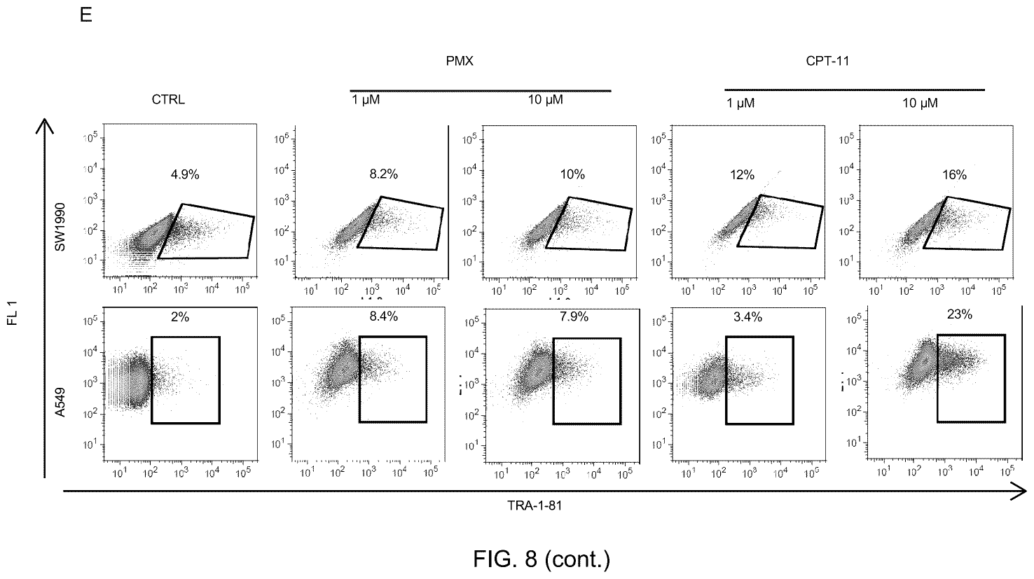

RESULTS. The common epitope of TRA-1-60 and TRA-1-81 antibodies is highly enriched in chemoresistant cancer cells. To develop a screening platform, a GEM-resistant (GR) cell line SW1990GR (FIG. 8A) was first established. The IC₅₀ for GEM of SW1990GR (59.4±5.3 nM) is ˜6 times higher than parental SW1990 cells (9.6±3.2 nM). Screening was then carried out to identify candidate markers correlated with chemoresistance of cancer cells (FIG. 1A). The parental line expressing an EBFP reporter (SW1990EBFP) and SW1990GR cells were mixed and stained with a human cell surface marker antibody library. The positive percentage of each marker in the two cell lines was quantified and further analyzed in two-dimensional space. As shown in FIG. 8B, the X axis represents the absolute difference value, obtained by subtracting the positive percentage in SW1990EBFP cells from that of SW1990GR cells; the Y axis indicates the fold change, obtained by dividing the positive percentage in SW1990GR cells by that of the SW1990EBFP cells. As highlighted, eight antigens were preferentially expressed in SW1990GR cells (FIG. 8B). Among them was CD24, a marker of pancreatic cancer stem cells and regulator of drug resistance in breast cancer cell lines, which validates our screening platform. After several rounds of validation, TRA-1-60 and TRA-1-81 expressing cells were confirmed as enriched in the SW1990GR cell line (FIG. 1B).

As shown in a previous report based on glycan array screening, TRA-1-60 and TRA-1-81 antibodies bind to the same polysaccharide epitope with high affinity. To confirm this correlation, SW1990 and SW1990GR cells were co-stained with TRA-1-60 and TRA-1-81 antibodies. The two antigens showed strong co-staining in flow cytometry and identify the same cell population, which is significantly enriched in SW1990GR cells (16.5%±2.7%) compared to SW1990 cells (2.8%±1.3%) (FIG. 1C, D). Therefore, the two markers interchangeably were used interchangeably for subsequent experiments.

To determine whether this epitope is enriched in other GR PDAC cell lines, AsPC-1GR, HPAF-IIGR and CaPan-2GR, and their corresponding parental cell lines were analyzed via flow cytometry. The TRA-1-60⁺/TRA-1-81⁺ cell population was significantly larger in all three GR cell lines compared with corresponding parental lines (FIG. 1E, F). To examine whether TRA-1-60 and TRA-1-81 are enriched in PDAC cells after acute treatment, SW1990 cells were treated with 50, 100 or 250 nM GEM for 5 days. Flow cytometry analysis indicated a significant increase of TRA-1-81⁺ cells in GEM-treated SW1990 cells (FIG. 1G and FIG. 8C). Other cancer cell lines A549, RKO, HT29, HCT116 and DU145 were examined. Consistent with the results in SW1990 cells, a significant increase of TRA-1-81⁺ cell population was detected after acute GEM treatment (FIG. 1H and FIG. 8D). If the TRA-1-60 and TRA-1-81 epitope is enriched in PDAC cells resistant to other chemotherapeutic agents was tested, after treating SW1990 cells with PMX or CPT-11 for 5 days. Similar to the effects of GEM, flow cytometry showed that the TRA-1-81⁺ cell population was highly enriched in CPT-11 or PMX treated SW1990 cells (FIG. 1I and FIG. 8E). Additionally, the A549 cell line also exhibited a significant enhancement of TRA-1-81⁺ portion after acute CPT-11 or PMX treatment (FIG. 1J and FIG. 8E).

Whether the epitope of TRA-1-60 and TRA-1-81 antibodies also enriches CCCs in vivo, with xenograft tumors in nude mice derived from SW1990 cells implanted subcutaneously, was examined. Mice carrying tumors were randomly separated into two groups: one was treated with 50 mg/kg GEM via intraperitoneal injection twice a week for 4 weeks; the other was treated with the same volume of PBS as control. After 3 passages in vivo (˜6 months), the control and GR xenograft tumors were analyzed with immunofluorescence (FIG. 1K). The percentage of TRA-1-60⁺ cells was significantly increased in the GR xenograft tumors (FIG. 1L). Together, these experiments demonstrate that TRA-1-60 and TRA-1-81 are surface markers that significantly enrich CCCs both in vitro and in vivo.

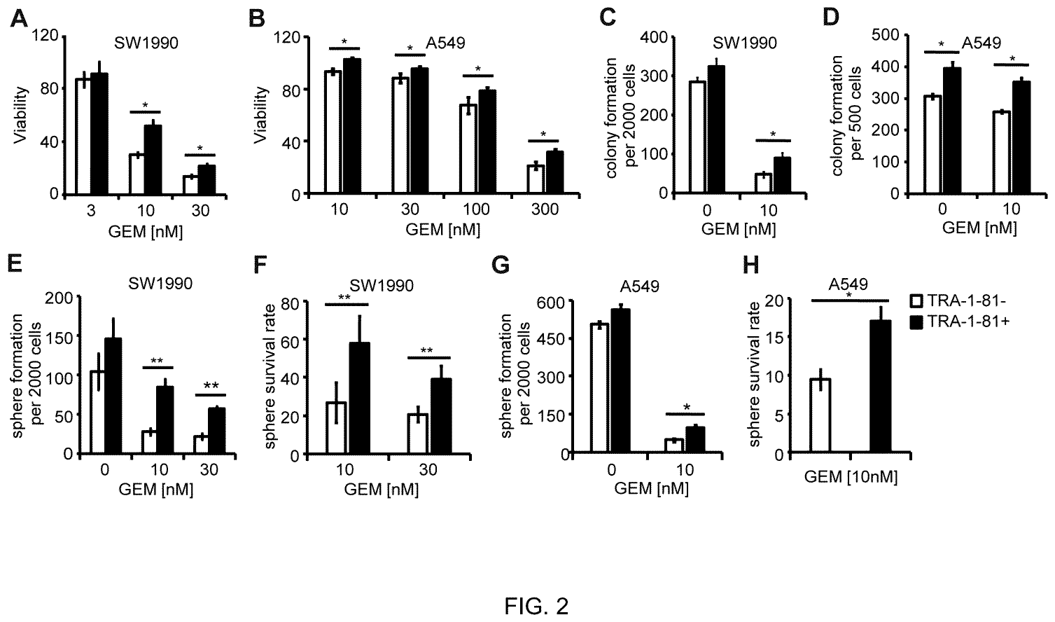

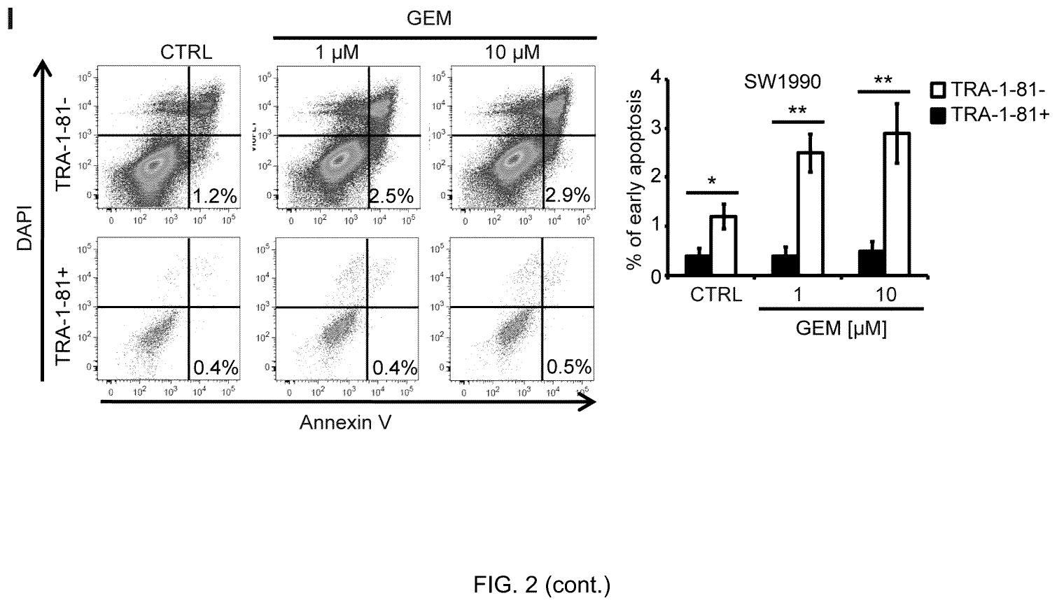

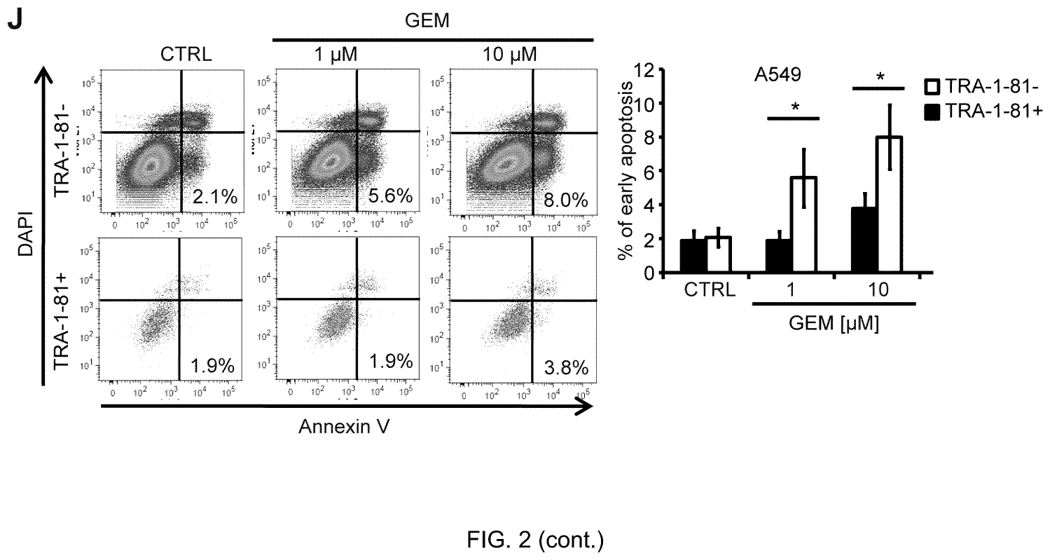

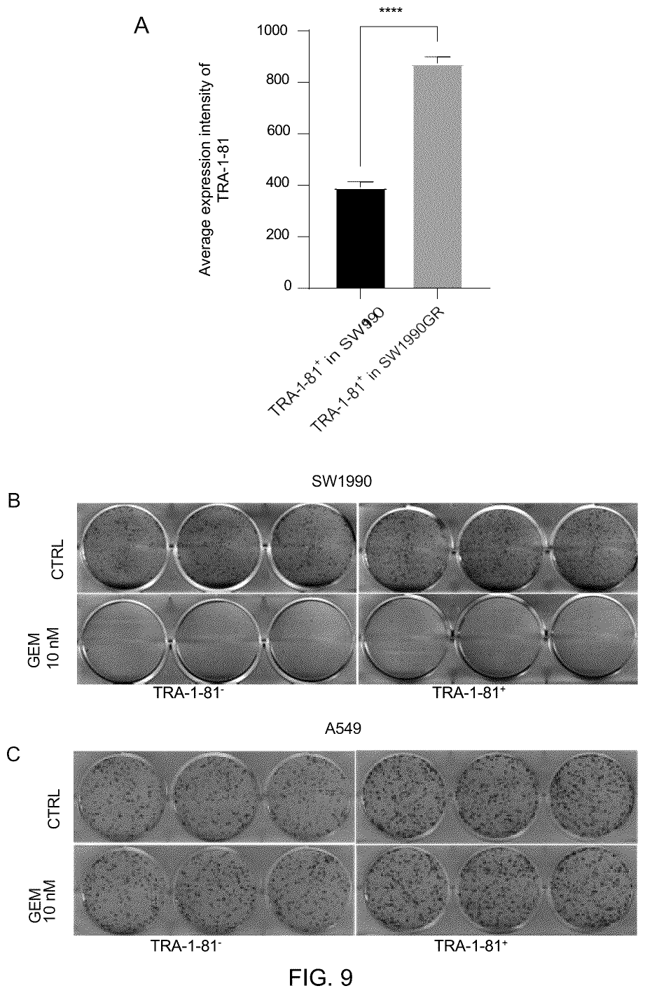

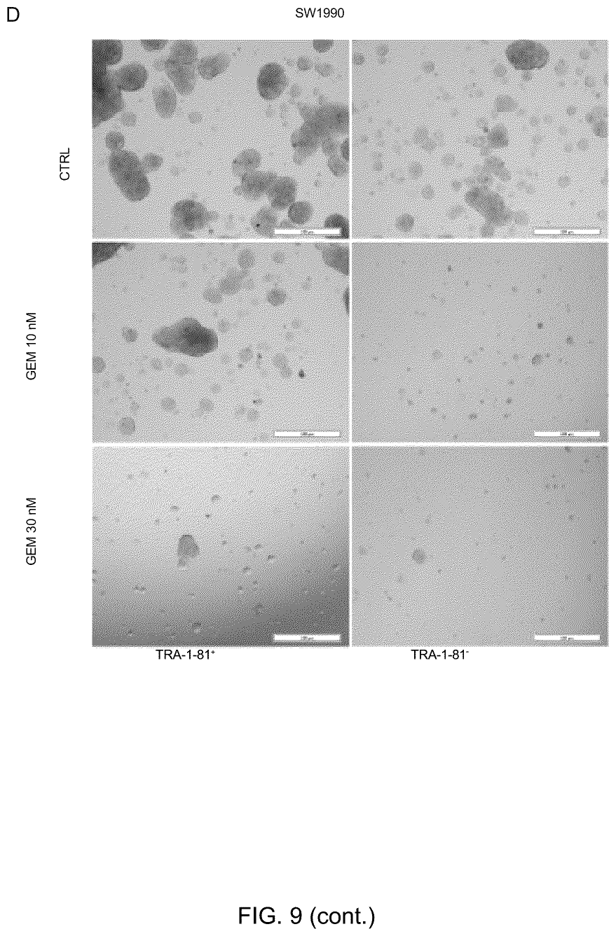

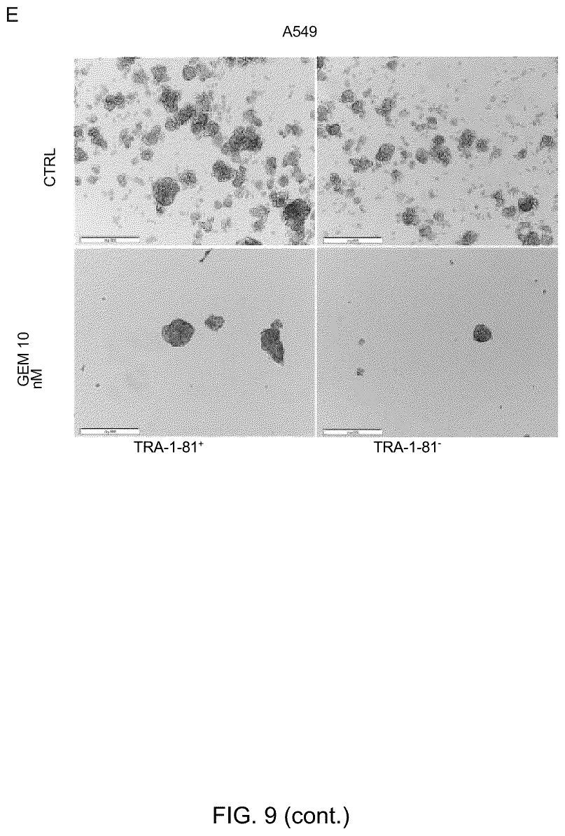

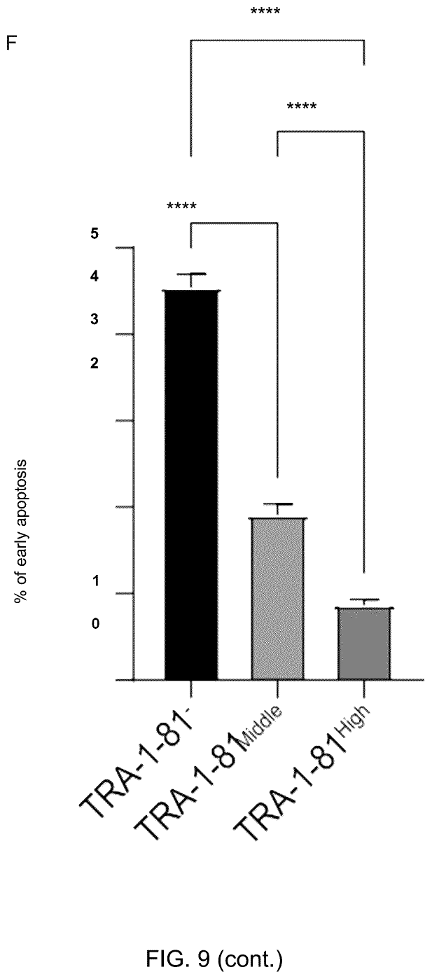

TRA-1-60⁺/TRA-1-81⁺ cells are resistant to gemcitabine treatment. TRA-1-60⁺/TRA-1-81⁺ cells were examined for survival, colony formation and sphere formation following GEM treatment. First, TRA-1-81⁺ and TRA-1-81⁻ cells isolated from the SW1990 cell line were treated with GEM at different concentrations (3, 10 or 30 nM) for 5 days. Relative cell viability was calculated by normalizing cell numbers in GEM-treated conditions to that in control conditions. TRA-1-81⁺ cells showed higher viability than TRA-1-81⁻ cells in 10 or 30 nM GEM (FIG. 2A). In addition, the TRA-1-81⁺ expression level of TRA-1-81⁺ SW1990GR cells is significantly higher than that of TRA-1-81⁺ SW1990 cells (FIG. 9A). Similarly, TRA-1-81⁺ A549 cells showed higher survival than TRA-1-81⁻ cells following GEM treatment (FIG. 2B). Second, colony formation was used to monitor single cell survival under GEM treatment. In both SW1990 and A549 cell lines, the TRA-1-81⁺ population yielded more colonies (FIG. 2C, D and FIG. 9B, C. Third, sphere formation capacity was evaluated in the presence of GEM. For both cell lines, more spheres were generated with TRA-1-81⁺ cells than TRA-1-81⁻ cells in both control and GEM treated conditions (FIG. 2E, G and FIG. 9D, E). Sphere survival rate was calculated by dividing sphere number in the presence of GEM by sphere number in control conditions. TRA-1-81⁺ cells showed significantly higher sphere survival rate than TRA-1-81⁻ cells (FIG. 2F, H).

Cell apoptosis induced by GEM was monitored with Annexin V staining. For both SW1990 cells (FIG. 2I) and A549 cells (FIG. 2J), the percentage of Annexin V⁺/DAPI⁻ (early apoptotic) cells was significantly higher in TRA-1-81⁻ populations in a GEM dose-dependent manner. Meanwhile, early apoptotic cells in the TRA-1-81⁺ population were modestly induced, which is consistent with resistance to chemotherapy drug treatment. Further, the early apoptosis rate was compared among TRA-1-81⁻, TRA-1-81^{medium} and TRA-1-81^{high} expressing populations in GR cells. The apoptotic rate in TRA-1-81^{high} cells is significantly lower than that of TRA-1-81^{medium} cells. (FIG. 9F).

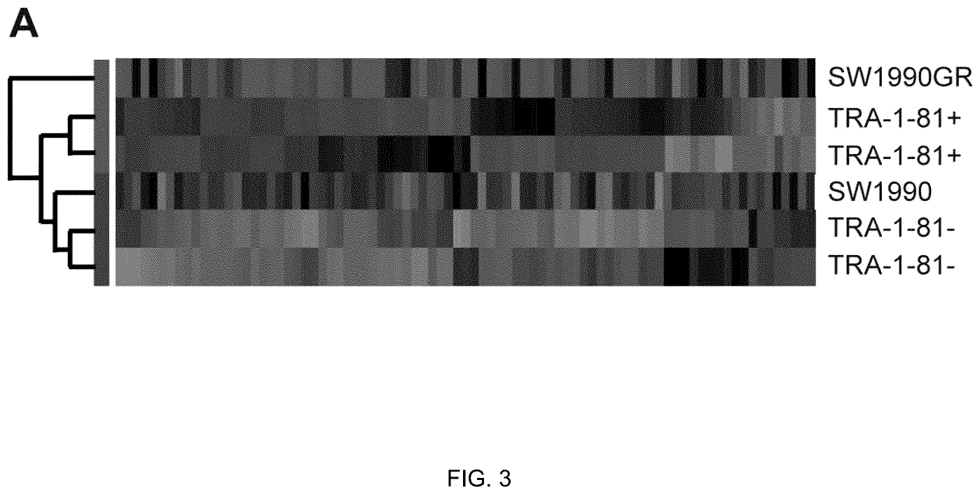

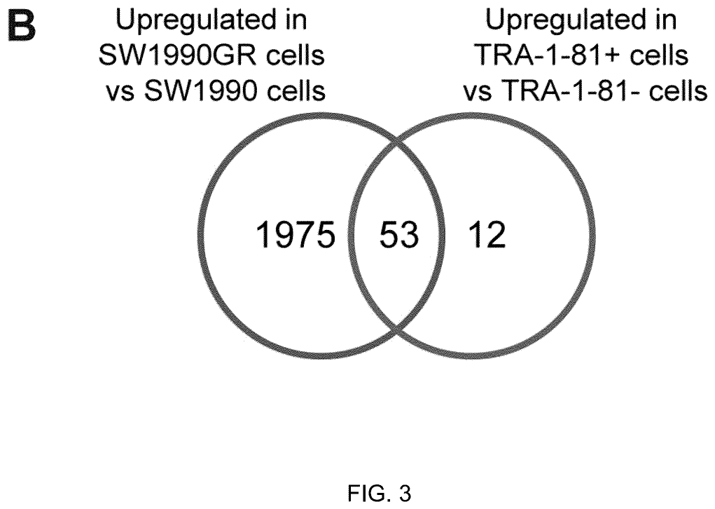

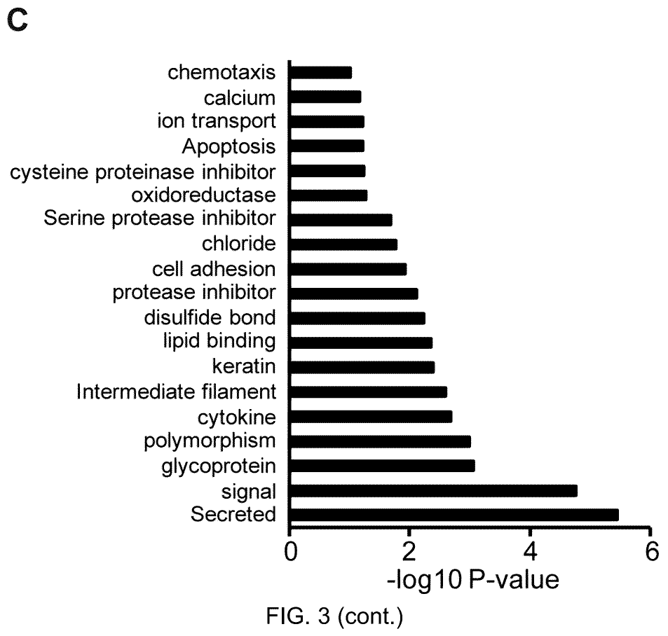

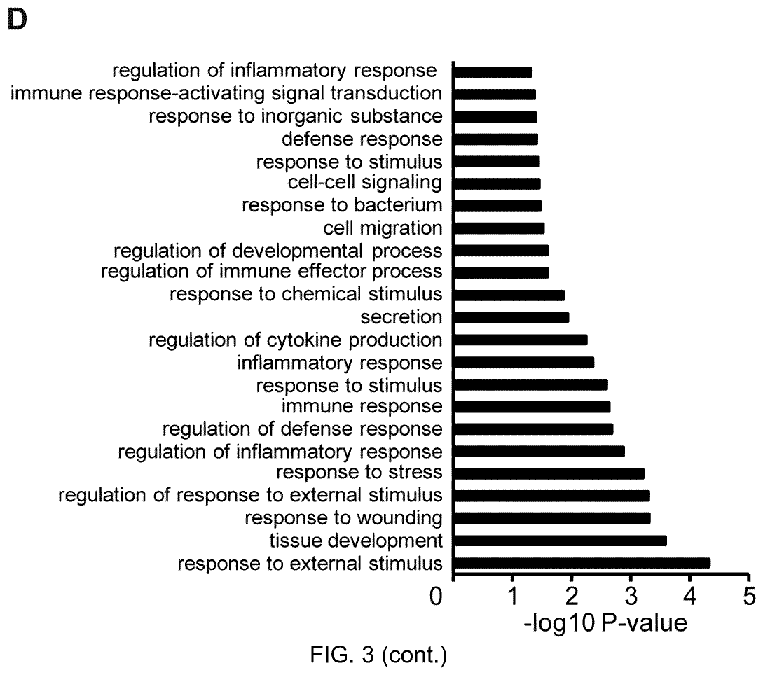

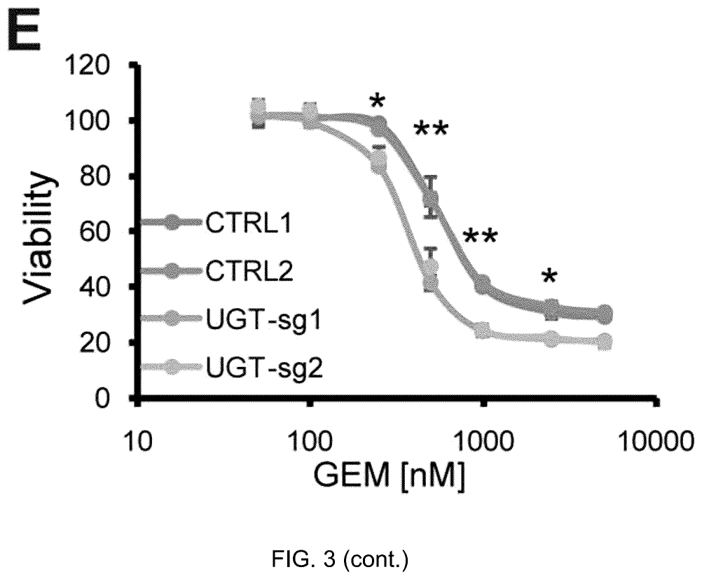

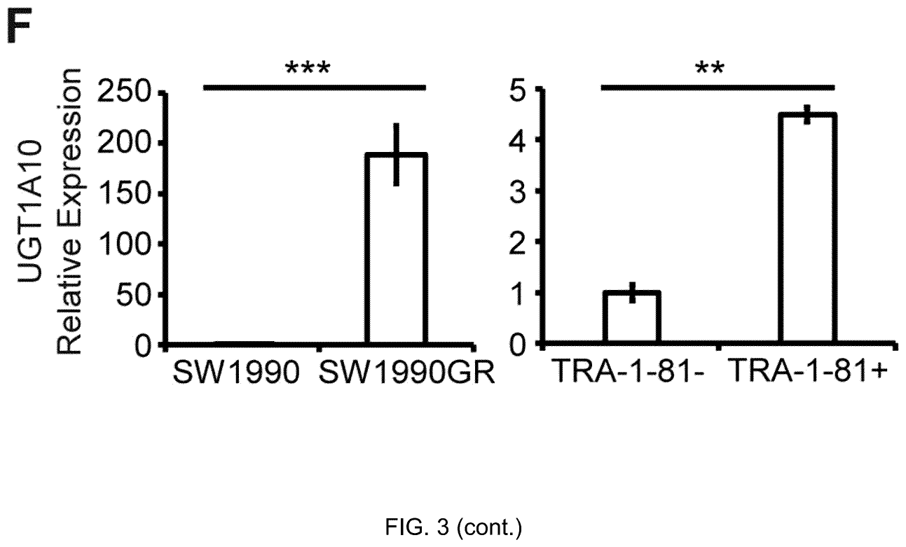

Gene expression profiling identifies UGT1A10 as a functional mediator of TRA-1-60/TRA-1-81 expression and chemoresistance. To interrogate the molecular basis for chemoresistance in TRA-1-60⁺/TRA-1-81⁺ cells, RNA-seq was used to compare the global transcriptome between TRA-1-81⁺ and TRA-1-81⁻ cells sorted from the SW1990 cell line, and independently, between parental SW1990 and SW1990GR cells. This identified 65 genes that are upregulated at least two-fold higher in TRA-1-81⁺ cells compared to TRA-1-81⁻ cells, and 2028 genes that are elevated at least three-fold in SW1990GR cells compared to parental SW1990 cells (FIG. 3A). The gene subset commonly up-regulated in both TRA-1-81⁺ and SW1990GR cells is comprised of 53 genes (FIG. 3B). Gene ontology analysis was performed to classify this subset into distinct groups according to protein category (FIG. 3C) and protein function (FIG. 3D). It was noted that glycoprotein is among the top group of proteins represented among these 53 shared genes (FIG. 3C). Most genes within this shared set are involved in response to stimulus, stress, and substrate (FIG. 3D), suggesting they may impact robustness of survival in TRA-1-81⁺ cells when challenged with chemo-drugs.

From the shared gene set, 7 candidates that showed relatively higher basal expression and higher fold change were selected for functional validation (Table 1). Two sgRNAs were designed for each gene to target the first coding exon, and two non-targeting sgRNAs were designed as controls (see experimental procedures). After transducing sgRNA and Cas9 enzyme by lentivirus, SW1990GR cells were examined for chemoresistance. The cells with UGT1A10 targeted (UGT-sg) showed significantly lower viability following GEM treatment (FIG. 3E), while targeting of the other 6 candidates showed no effect. Efficient reduction of UGT1A10 expression in UGT-sg cells was confirmed by western blotting experiments. Data from qRT-PCR experiments confirmed that UGT1A10 mRNA levels are strikingly higher in SW1990GR and TRA-1-81⁺ cells (FIG. 3F).

TABLE 1
Candidate genes for functional validation. Related to FIG. 3.

| | | Experiment 1 | | | Experiment 2 | | | | | |
|---|---|---|---|---|---|---|---|---|---|---|
| | | expression level | | | expression level | | | acquired chemoresistance | | |
| | | TRA- | TRA- | | TRA- | TRA- | | expression level | | |
| ID | name | 1-81− | 1-81+ | fold | 1-81− | 1-81+ | fold | SW1990 | SW1990 GR | fold |
| NM_020041 | SLC2A9 | 1.21 | 3.37 | 2.78 | 1.62 | 4.34 | 2.68 | 0.19 | 11.22 | 58.09 |
| NM_001033723 | ZNF704 | 0.27 | 0.77 | 2.80 | 0.22 | 0.60 | 2.75 | 0.03 | 3.04 | 114.15 |
| NM_005555 | KRT6B | 0.43 | 2.29 | 5.27 | 0.31 | 1.12 | 3.58 | 0.06 | 5.67 | 102.11 |
| NM_019075 | UGT1A10 | 0.45 | 1.54 | 3.44 | 0.31 | 1.21 | 3.89 | 0.59 | 98.58 | 167.22 |
| NM_006072 | CCL26 | 0.12 | 0.47 | 3.96 | 0.07 | 0.27 | 4.15 | 0.19 | 35.60 | 188.56 |
| NM_015090 | NFASC | 0.08 | 0.33 | 4.27 | 0.04 | 0.18 | 4.15 | 0.01 | 4.44 | 337.58 |
| NM_001072 | UGT1A6 | 0.66 | 6.69 | 10.09 | 0.62 | 6.36 | 10.29 | 0.00 | 57.10 | 1.99E+05 |

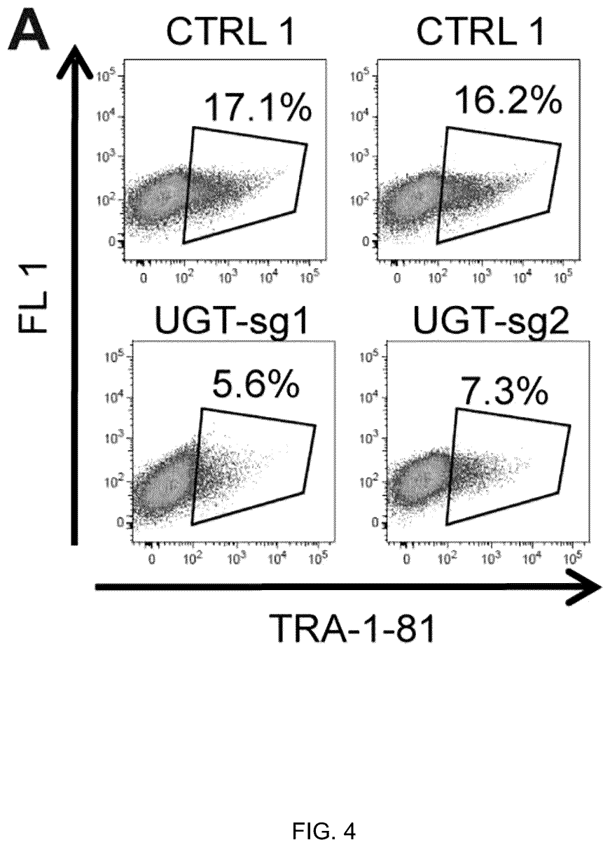

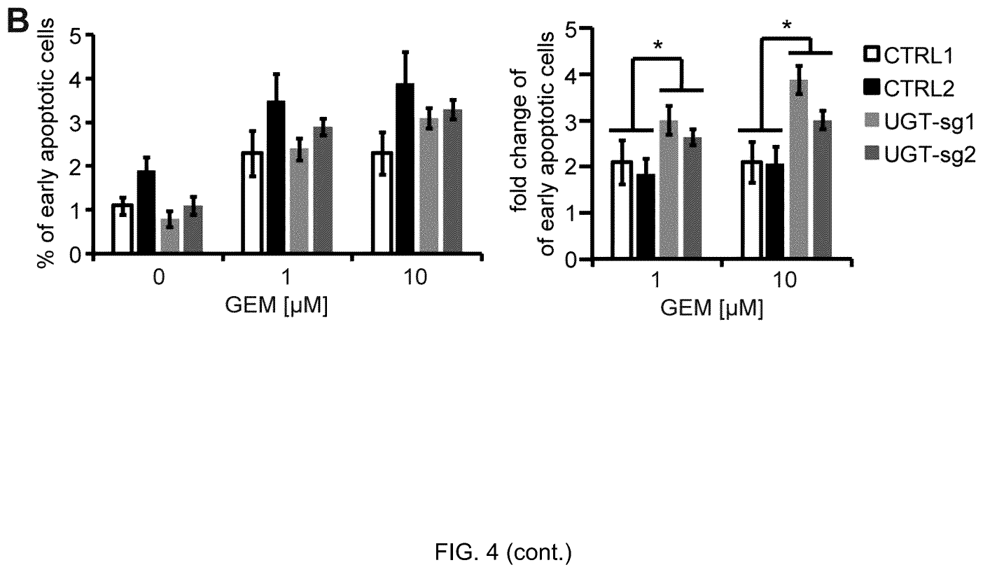

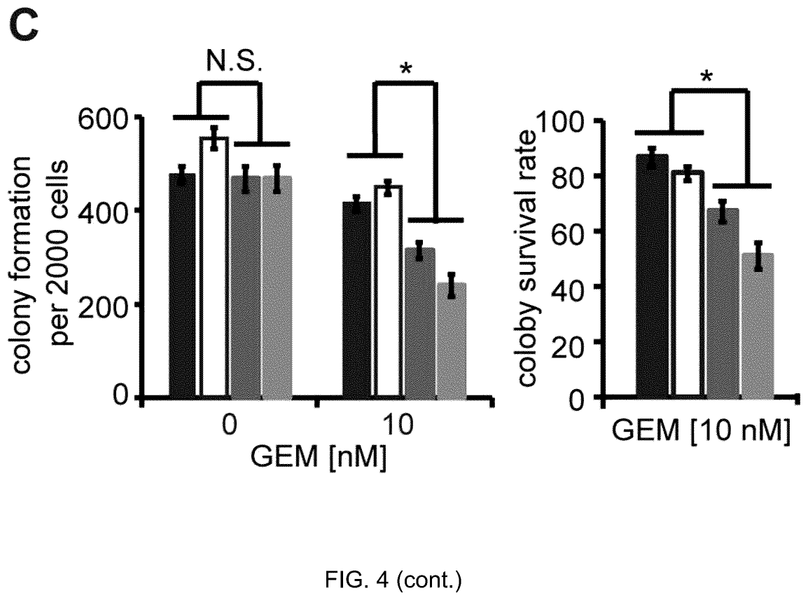

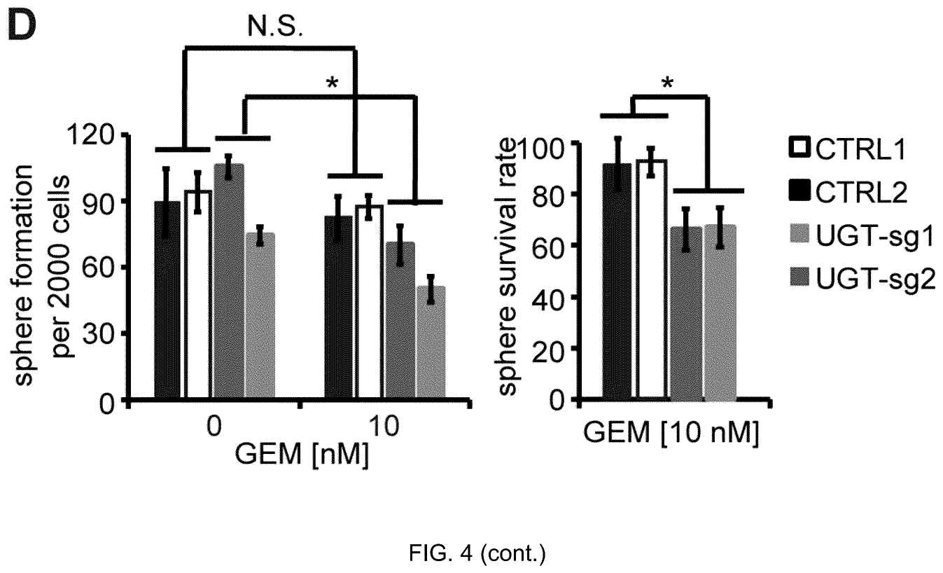

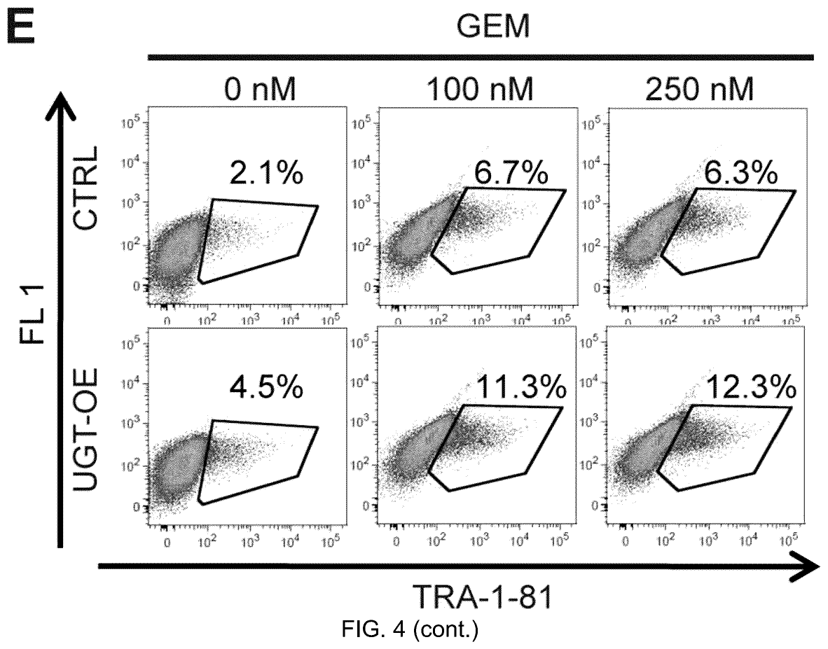

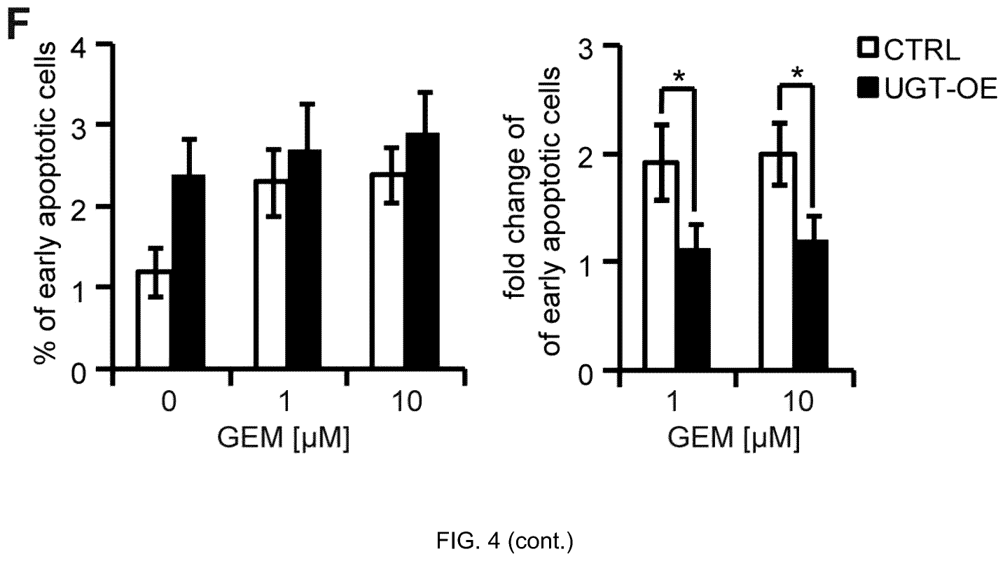

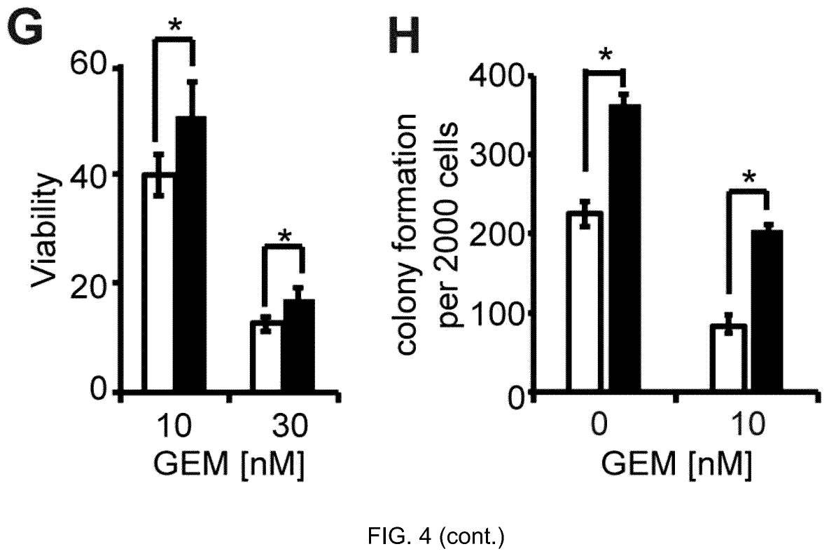

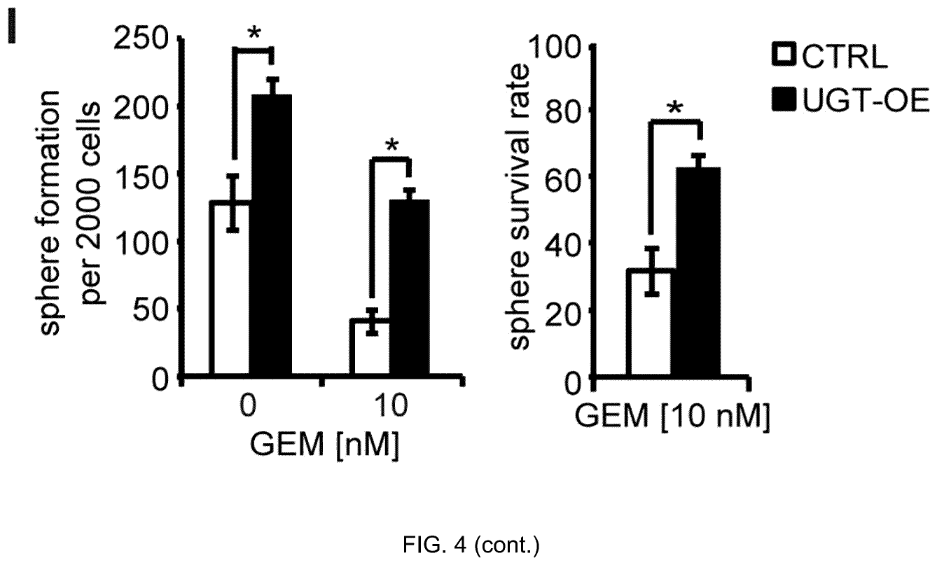

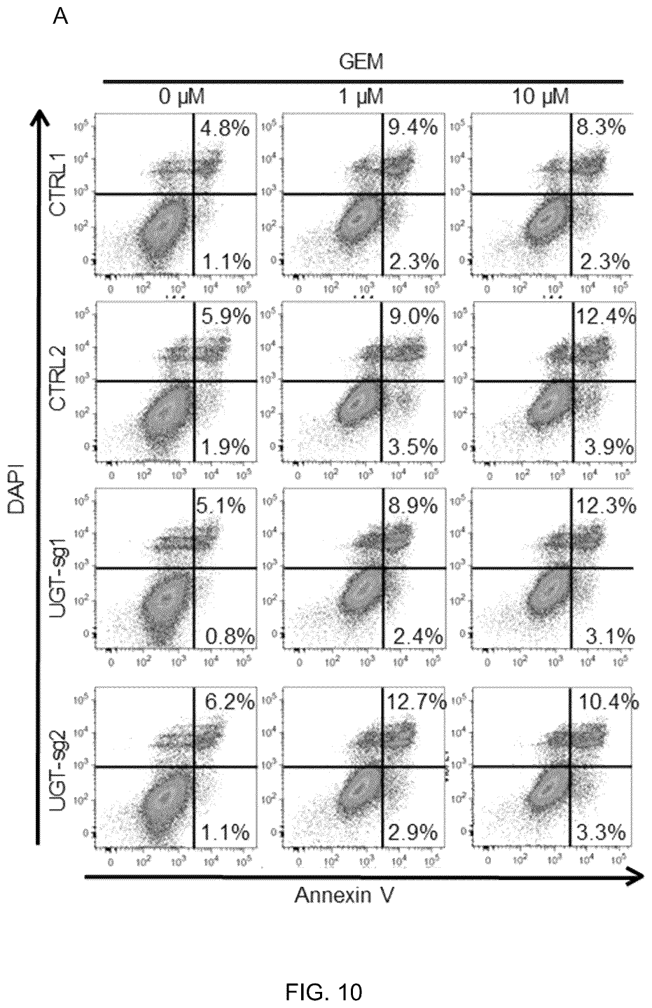

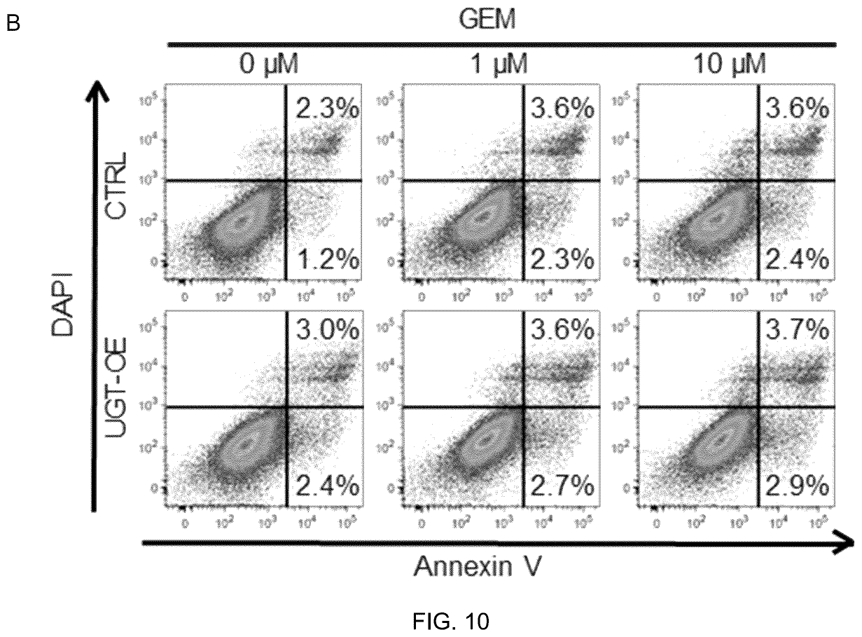

UGT1A10 is both required and sufficient to maintain high levels of TRA-1-60⁺/TRA-1-81⁺ cells and enhance chemoresistance. Flow cytometry analysis showed that the percentage of TRA-1-81⁺ cells was significantly decreased in UGT-sg cells, suggesting that UGT1A10 is required to maintain TRA-1-81 expression (FIG. 4A). GEM sensitivity was monitored by comparing the percentage of Annexin V⁺/DAPI⁻ early apoptotic cells induced by GEM-treatment with that in control treatment. UGT-sg cells showed higher sensitivity to GEM-induced cell apoptosis than control SW1990GR cells (FIG. 4B and FIG. 10A). Colony-forming capacity was also compared. In GEM-free conditions, there is no significant difference between UGT-sg cells and control SW1990GR cells. In the GEM-treated conditions, colony numbers derived from UGT-sg cells are significantly lower compared to control cells (FIG. 4C, left panel). The colony survival rate was determined by normalizing colony number in the presence of GEM with the number in the absence of GEM, and UGT-sg cells showed a significantly decreased colony survival rate (FIG. 4C, right panel), suggesting that knockout of UGT1A10 impaired the robustness of colony-forming capacity. Finally, the UGT-sg and control SW1990GR cells were cultured in suspension to examine their relative sphere forming ability. The number of spheres produced by UGT-sg cells decreased dramatically in the presence of GEM. In contrast, the number of spheres produced by the control cells was not significantly changed between GEM and control conditions (FIG. 4D). The sphere survival rate of UGT-sg cells was significantly lower than that of control cells. Therefore, UGT1A10 is necessary both for the maintenance of TRA-1-81⁺ cells, as well as chemoresistance.

To determine whether UTG1A10 is sufficient to induce TRA-1-81 expression and chemoresistance, UGT1A10 was overexpressed ectopically in SW1990 cells (UGT-OE). Cells expressing firefly luciferase were used as control (CTRL). A significant increase of TRA-1-81⁺ cells was detected in UGT-OE compared with control cells, either in the absence or presence of GEM (FIG. 4E). While GEM treatment significantly increased the percentage of Annexin V⁺/DAPI⁻ early apoptotic cells (FIG. 4F and FIG. 10B), UGT-OE cells did not respond to GEM-induced cell apoptosis (FIG. 4F, right). Indeed, the viability of UGT-OE cells is higher than control cells following GEM treatment (FIG. 4G). Furthermore, UGT-OE cells showed less reduction of colony-forming (FIG. 4H) and sphere-forming capacity (FIG. 4I) after challenge by GEM. In summary, UGT1A10 is both required and sufficient to enhance TRA-1-60 and TRA-1-81 expression and chemoresistance.

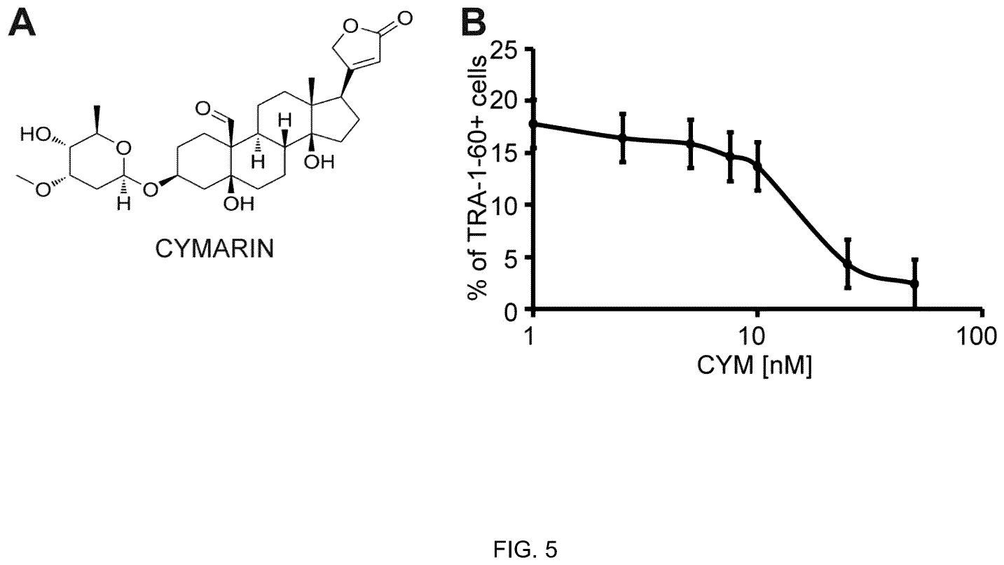

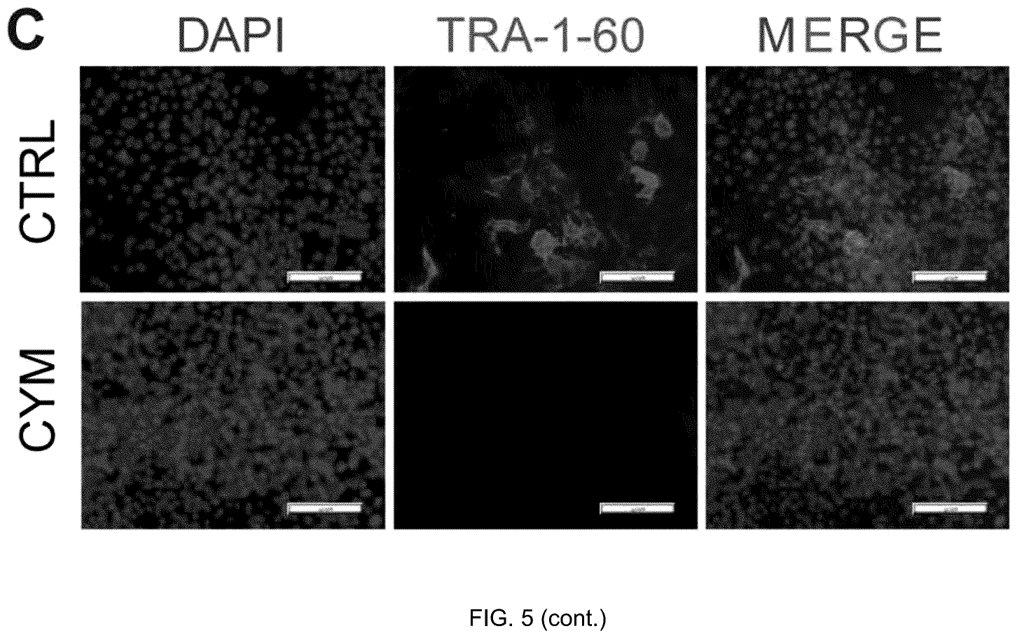

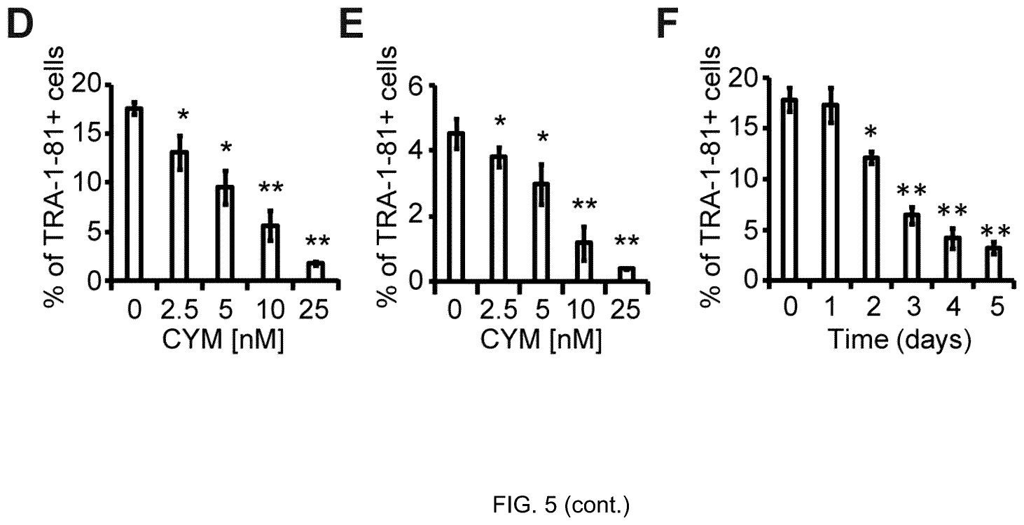

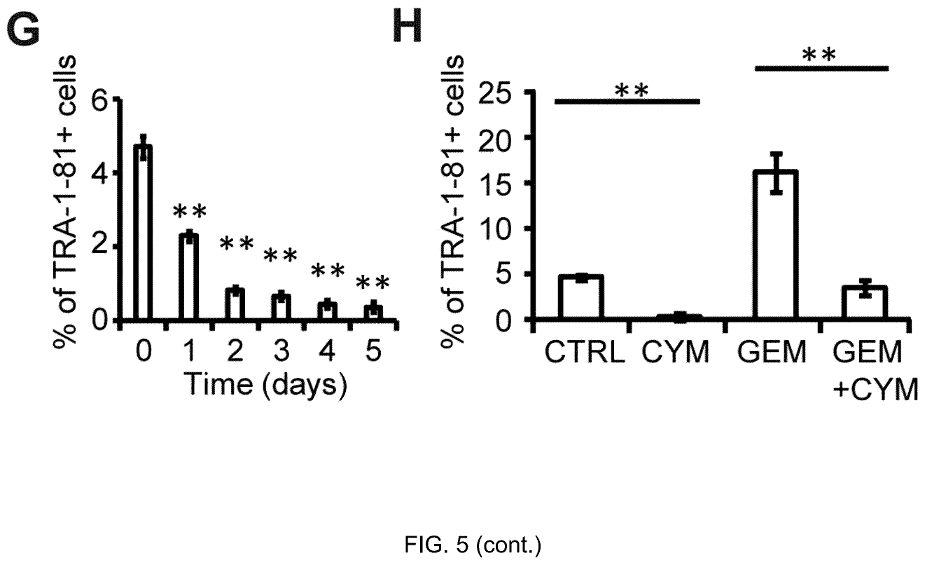

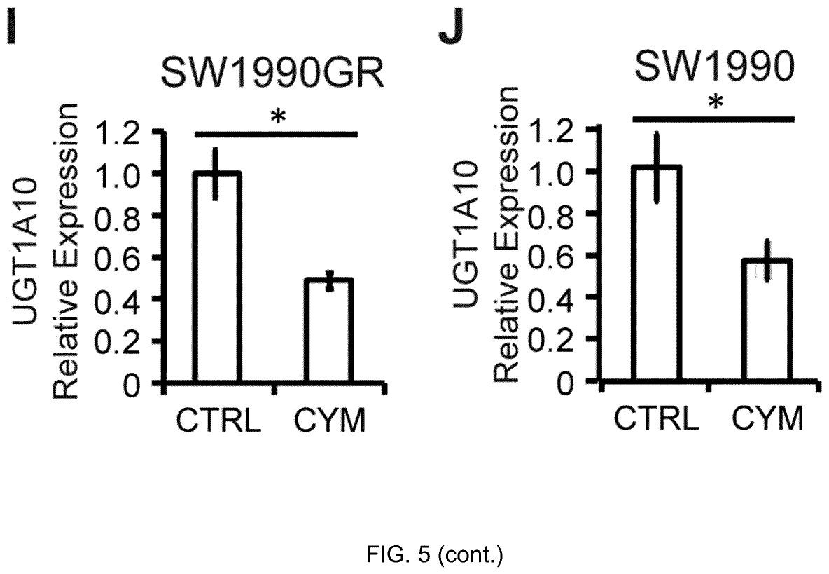

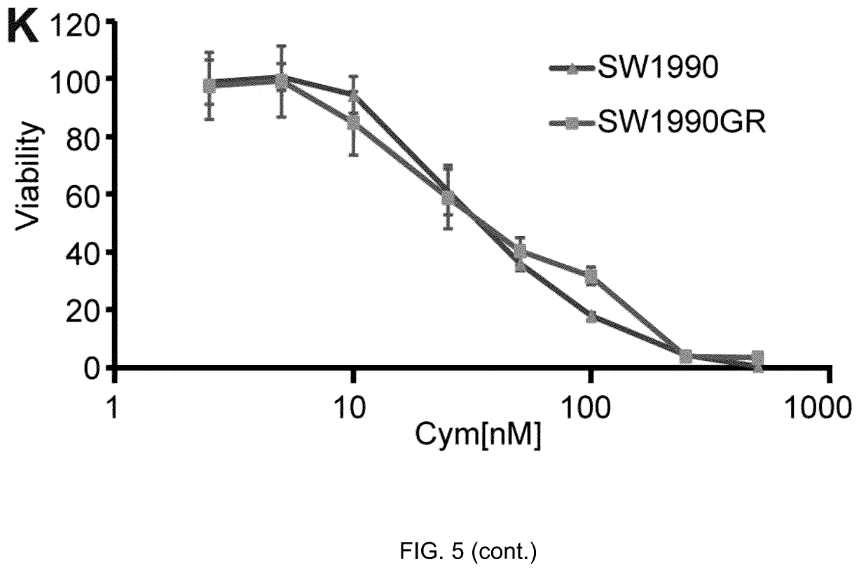

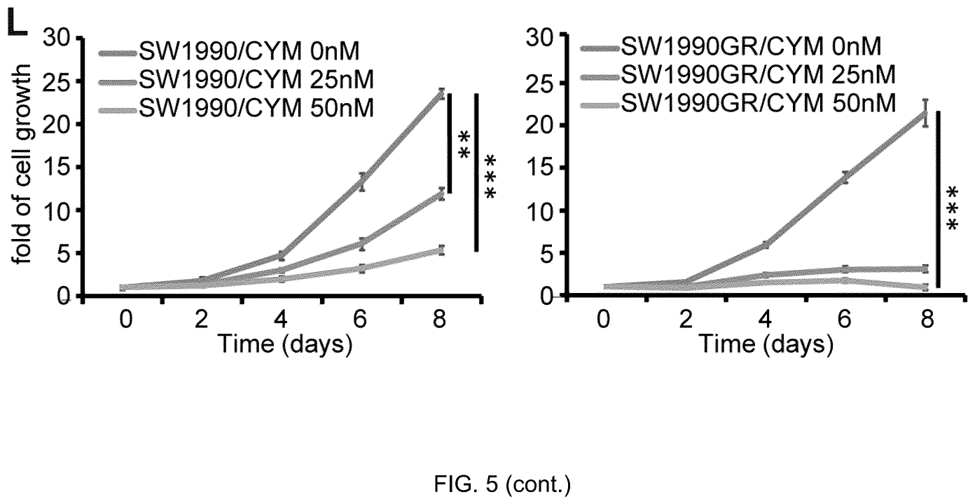

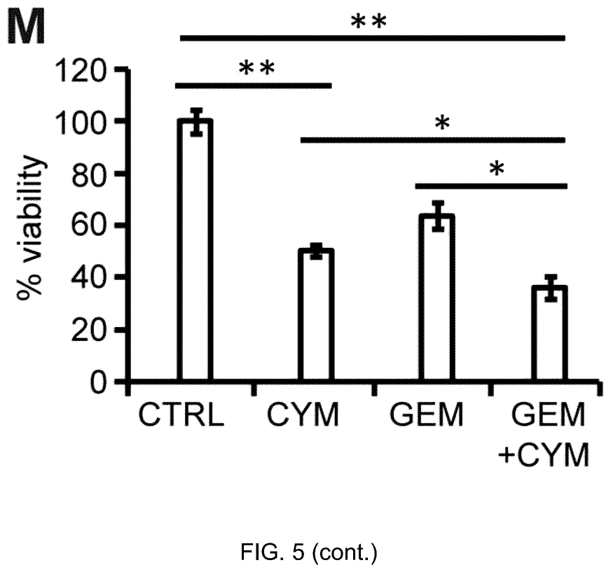

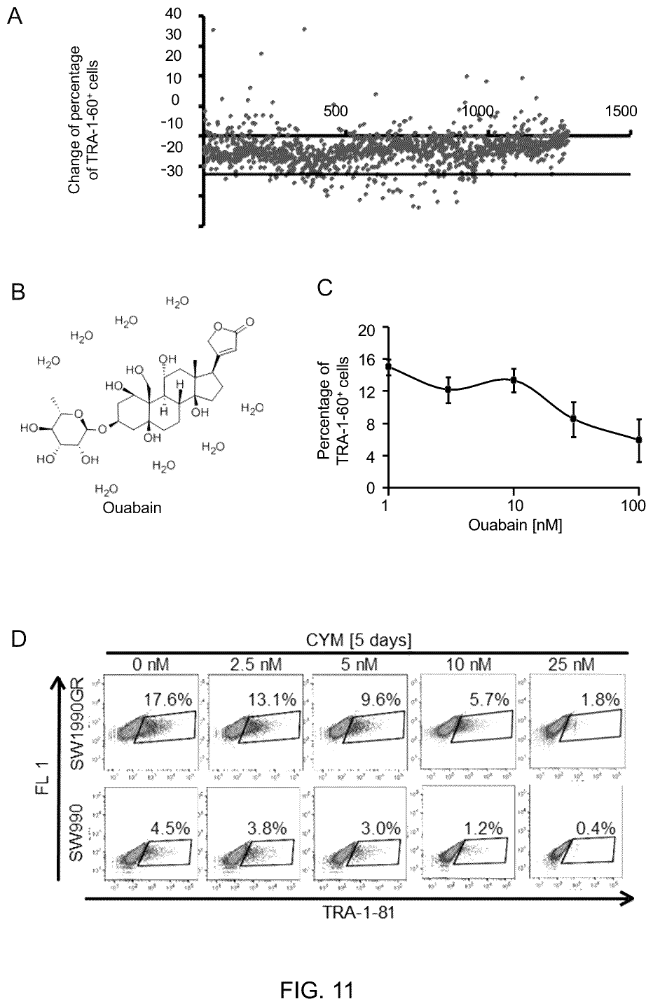

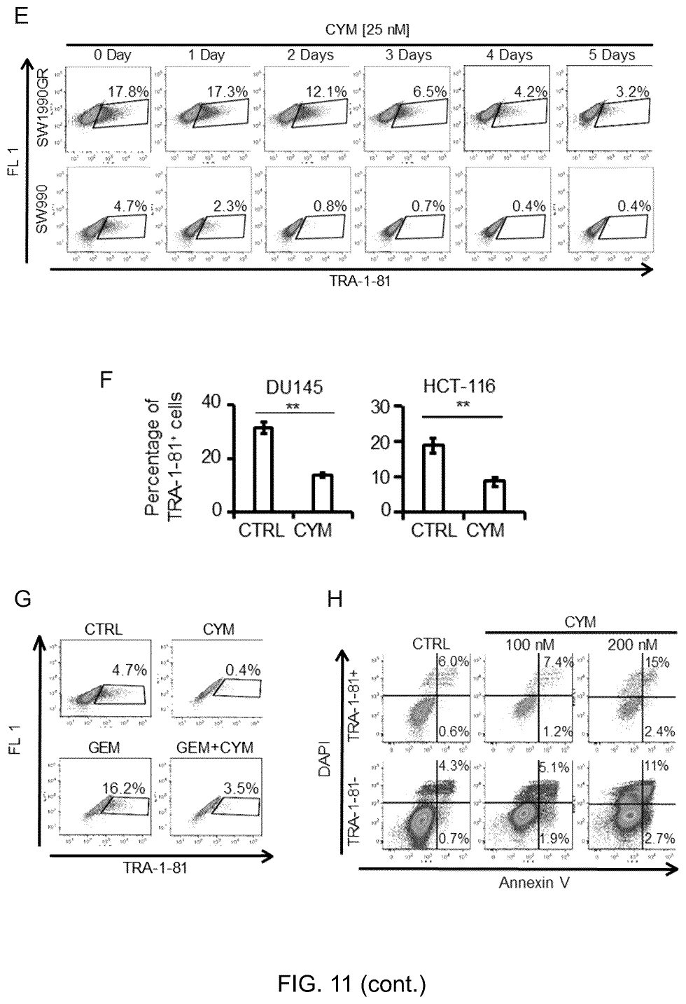

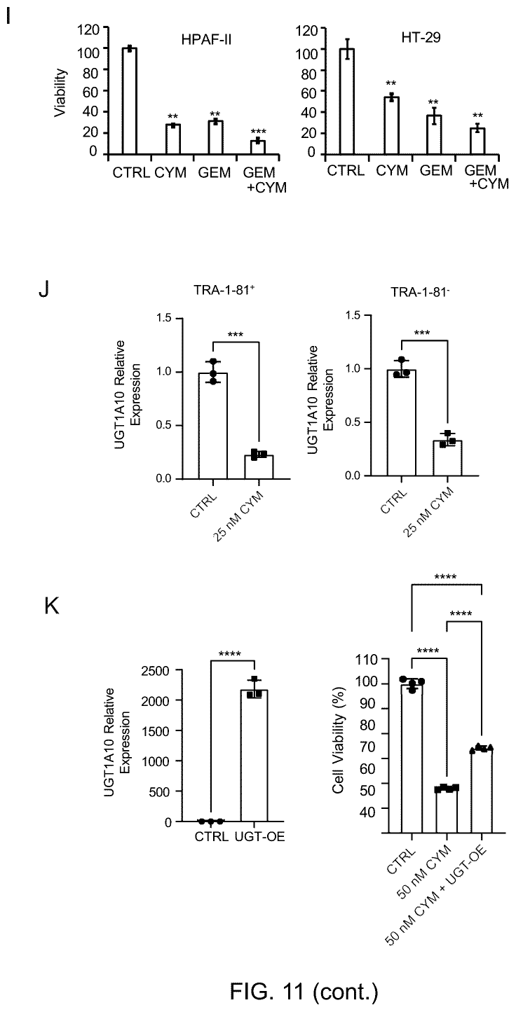

A high content chemical screen identifies Cymarin as a compound that specifically eliminates TRA-1-60⁺/TRA-1-81⁺ cells and increases chemosensitivity in vitro. A high content chemical screen was performed to identify drug candidates specifically eliminating TRA-1-60⁺/TRA-1-81⁺ cells. Since this antigen appears to mark CCCs, such a drug could possibly reduce chemoresistance. After treating SW1990GR cells with the library, the percentage of TRA-1-60⁺ cells was assessed with immunocytochemistry. 20 compounds that decreased the percentage of TRA-1-60⁺ cells more than 12% were picked as primary hits (FIG. 11A). Two of the hits were able to be validated, Cymarin (CYM) (FIG. 5A) and the structurally related Ouabain (FIG. 11B). CYM was focused on since it showed higher potency than Ouabain (FIG. 11C).

CYM decreased the percentage of TRA-1-60⁺ cells in a dose-dependent manner based on immunofluorescent staining, with an IC₅₀=15.2±4.3 nM (FIG. 5B). After 5 days treatment with 25 nM CYM, the percentage of TRA-1-60⁺ cells decreased from ˜18% to less than 5% (FIG. 5B, C). Flow cytometry analysis confirmed that CYM decreased the percentage of TRA-1-81⁺ SW1990GR cells in a dose-dependent manner, with IC₅₀=5.1±1.5 nM (FIG. 5D and FIG. 11D). For the parental SW1990 cells, CYM also repressed TRA-1-81⁺ cells in a dose-dependent manner, with IC₅₀=6.3±2.2 nM (FIG. 5E and FIG. 11D). To determine the minimal time required to eliminate TRA-1-81⁺ cells, a time course experiment was performed and showed a decrease of TRA-1-81⁺ cells over time in both SW1990GR cells (FIG. 5F and Supplementary FIG. 5D) and SW1990 cells (FIG. 5G and FIG. 11E). CYM also decreased the percentage of TRA-1-81⁺ cells in other cancer cell lines, for example, prostate cancer cell line DU145 (FIG. 11F, left), and colorectal cancer cell line HCT-116 (FIG. 11F, left). To determine whether CYM attenuates the enrichment of TRA-1-81⁺ cells caused by acute GEM treatment, the percentage of TRA-1-81⁺ cells was evaluated by flow cytometry in SW1990 cells after 5 days treatment with 100 nM GEM, 25 nM CYM, or 100 nM GEM plus 25 nM CYM. CYM treatment decreased the percentage of TRA-1-81⁺ cells effectively regardless of GEM (FIG. 4H and FIG. 11G).

Whether or not CYM may eliminate of TRA-1-81⁺ cells either by killing TRA-1-81⁺ cells or through suppression of TRA-1-60/TRA-1-81 epitope expression was considered. After one day treatment of 100 nM or 200 nM CYM, apoptosis was assessed in SW1990GR cells. The percentage of Annexin V⁺/DAPI⁻ (early apoptotic) cells and Annexin V⁺/DAPI⁺ (late apoptotic) cells were increased in both TRA-1-81⁺ and TRA-1-81− subpopulations in a dose-dependent manner (FIG. 11H). However, the increasing trend of apoptosis showed little difference between TRA-1-81⁺ and TRA-1-81⁻ populations. Rather, qRT-PCR assays showed UGT1A10 transcript levels were decreased with CYM treatment in both SW1990GR (FIG. 5I) and SW1990 (FIG. 5J) cells. Therefore, most likely the reduction of TRA-1-60⁺/TRA-1-81⁺ cells caused by CYM is not due to targeted cell death, but due to modulation of functional molecular targets, for example levels of UGT1A10.

To evaluate the effect of CYM on cell viability, both SW1990 and SW1990GR cells were cultured with CYM (concentration ranging 0-500 nM) for 3 days. Viability of both cell lines was decreased in a dose-dependent manner, with IC₅₀=33.8±2.6 nM for SW1990 cells and 40.8±3.1 nM for SW1990GR (FIG. 5K). Proliferation of both cell lines was monitored with MTT in 0, 25 or 50 nM CYM. 50 nM CYM significantly decreased the proliferation of SW1990 cells (FIG. 5L, left), while 25 nM CYM was sufficient to block the proliferation of SW1990GR cells (FIG. 5L, right). SW1990 cells were then treated with 30 nM GEM, 25 nM CYM or 30 nM GEM plus 25 nM CYM for 3 days. Cells exposed to the combined treatment showed significantly lower viability than cells treated with single agents (FIG. 5M). Likewise, CYM significantly decreased the survival of GEM-resistant cells of another PDAC cell line, HPAF-II (FIG. 11I, left), and a colorectal cancer cell line, HT-29 (FIG. 11I, right). Cymarin significantly inhibits the expression of UGT1A10 in both TRA-1-81⁺ and TRA-1-81⁻ subpopulations (FIG. 11J). Importantly, the ectopic expression of UGT1A10 partially rescues the Cymarin anti-chemoresistant activity (FIG. 11K). Together, CYM represses UGT1A10 expression levels (FIGS. 5I and 5J), reduces the expression of TRA-1-60/TRA-1-81 (FIG. 5H), and, at least partially, eliminates chemoresistant cells (FIG. 5K).

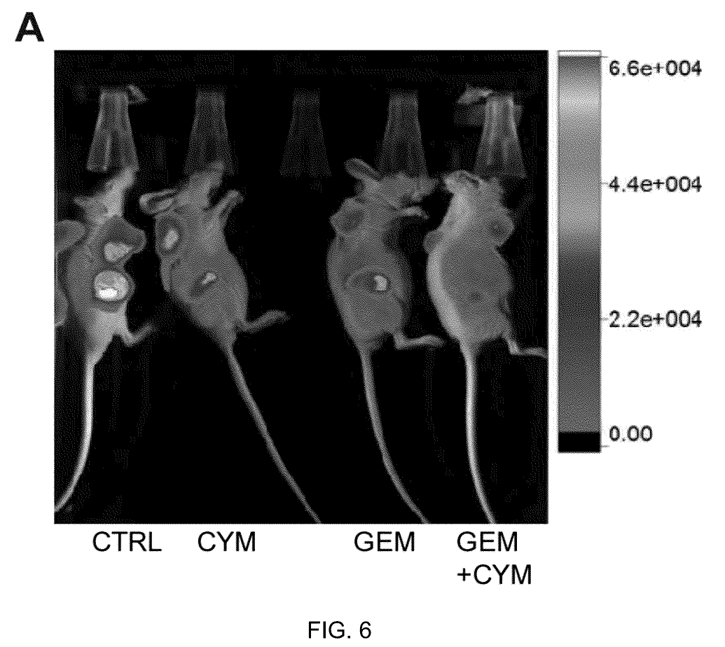

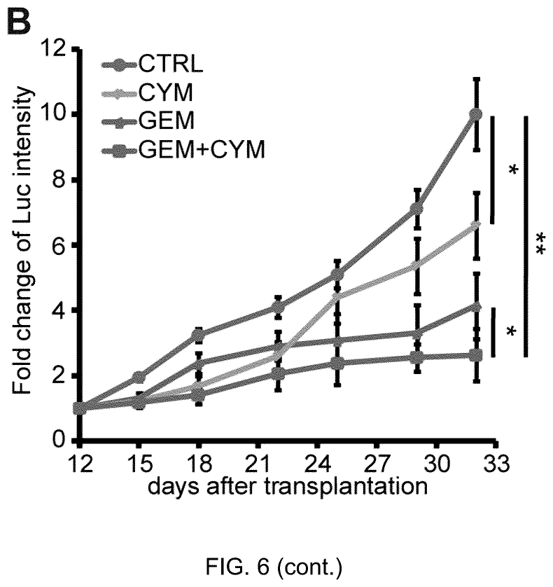

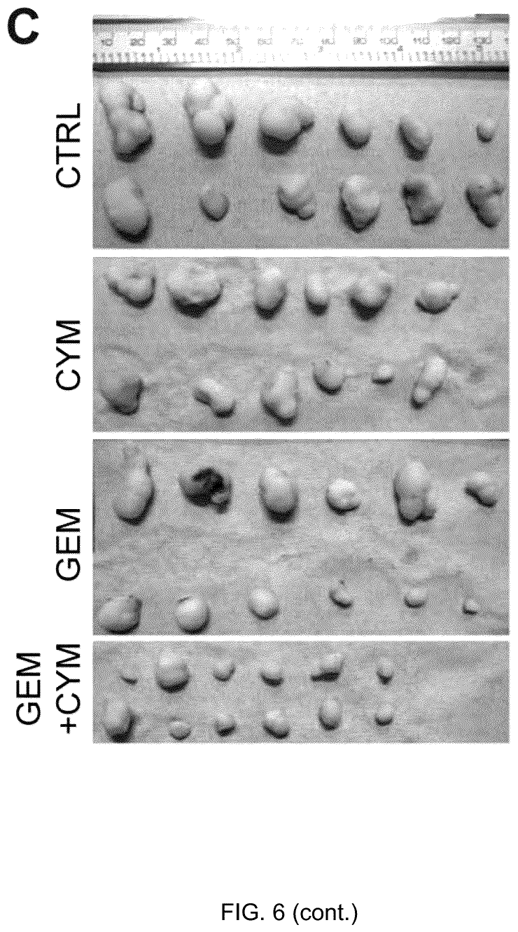

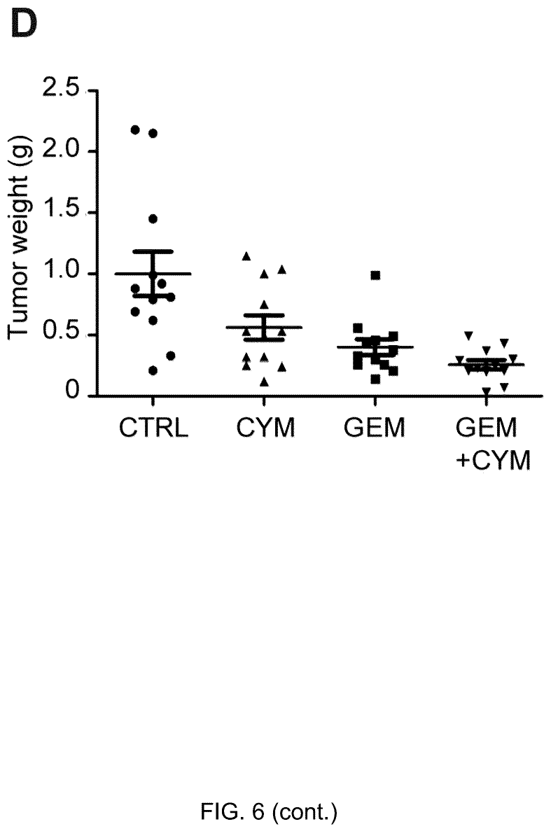

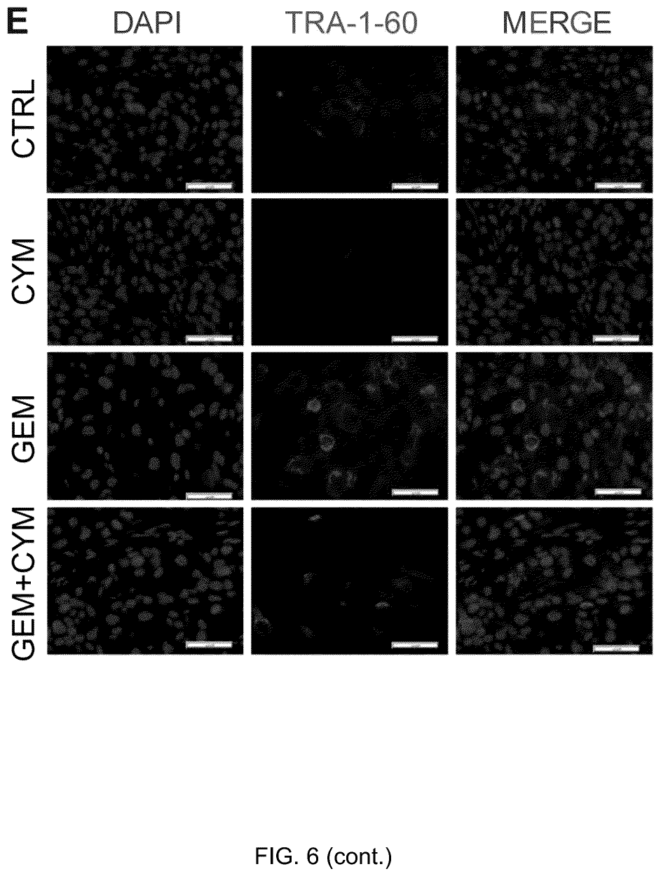

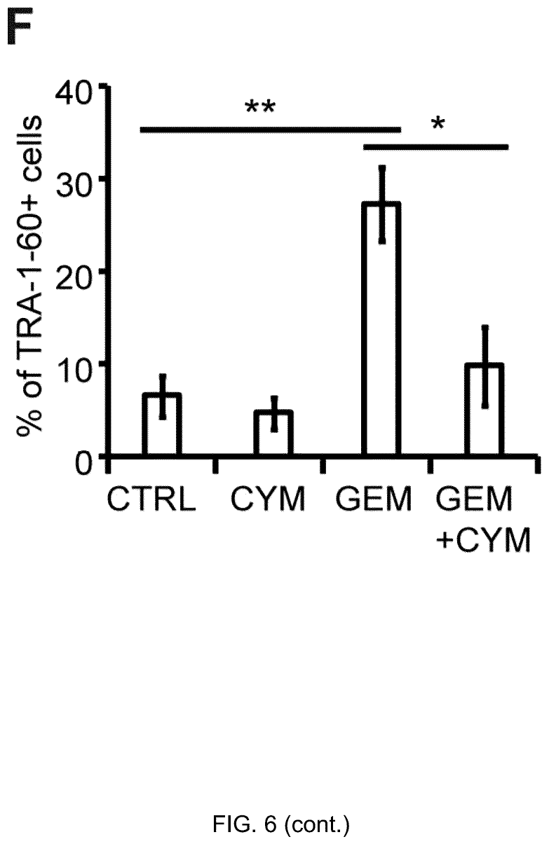

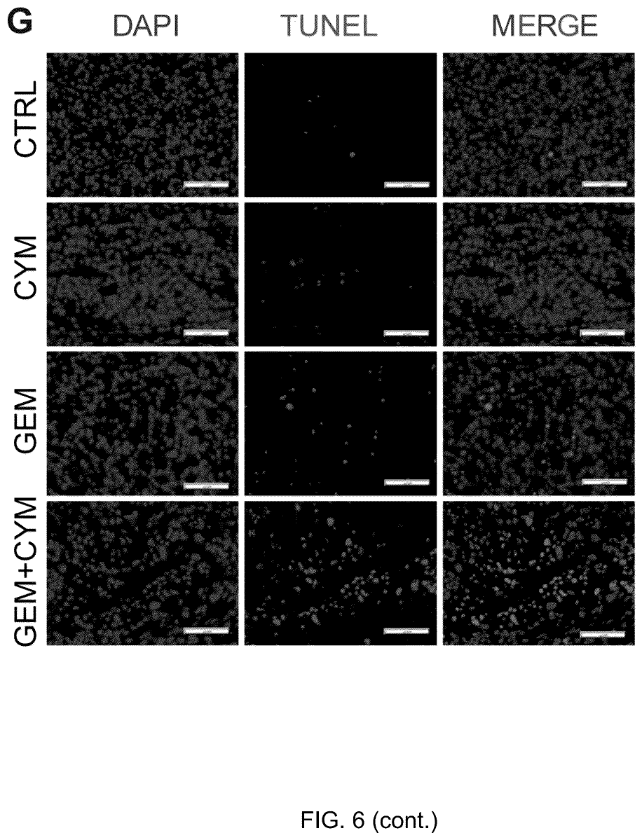

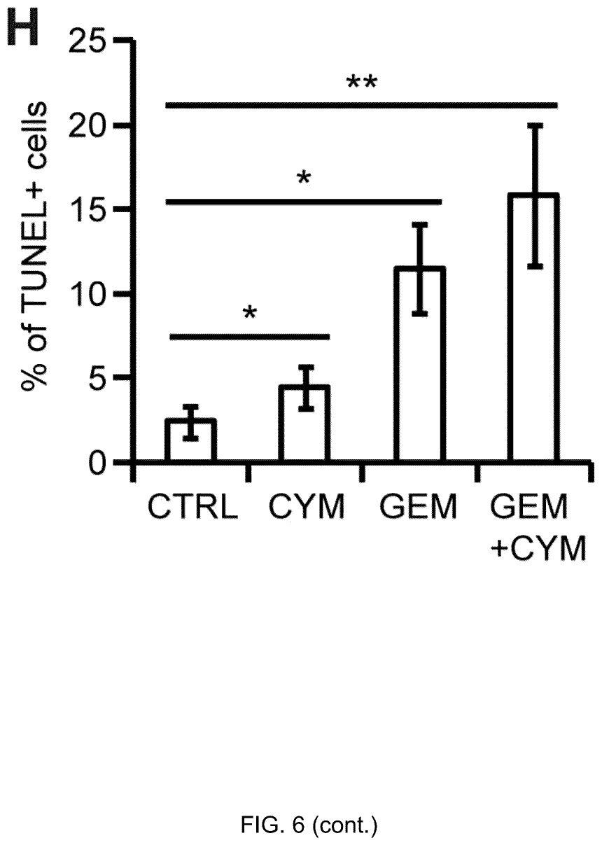

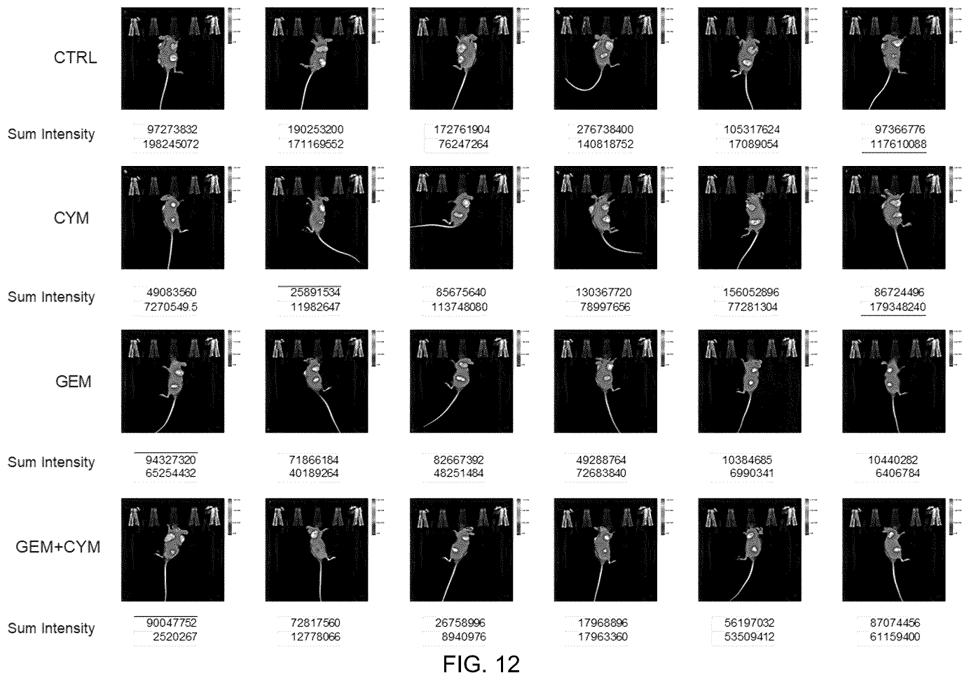

CYM suppresses TRA-1-60 expression and blocks tumor growth in vivo. Next it was sought to validate the effects of CYM in vivo, using a xenograft model with luciferase-labeled SW1990 cells propagated in nude mice. Tumor-harboring mice were separated into 4 groups and treated with PBS (control), 2 mg/kg CYM, 50 mg/kg GEM, or 2 mg/kg CYM plus 50 mg/kg GEM through intraperitoneal injections twice per week. Luciferase intensity was quantified twice per week and normalized with the value obtained from the starting day. After 3 weeks, tumors grew more slowly in mice treated with GEM or CYM than the control mice, while GEM plus CYM significantly retarded tumor growth compared to all other mice (FIG. 6A, B and FIG. 12). Consistent with imaging, of the four groups, mice treated with GEM plus CYM generated xenograft tumors with the smallest size (FIG. 6C) and weight (FIG. 6D).

The effects of CYM on TRA-1-60⁺ cells in vivo through examining the percentage of TRA-1-60⁺ cells in xenograft tumors with immunofluorescence was also tested. Consistent with the in vitro data, CYM administration decreased the percentage of TRA-1-60⁺ cells in GEM-treated tumors from ˜27% to ˜13% (FIG. 6E, F). The rate of cell apoptosis in xenograft tumors was assessed with TUNEL assays (FIG. 6G, H), which showed cell death increased in CYM treated tumors. Additionally, the percentage of TUNEL⁺ cells in tumors exposed to combined treatment was significantly higher than in tumors treated with GEM or CYM alone. In summary, CYM eliminates TRA-1-60⁺ cells and increases chemosensitivity in vivo.

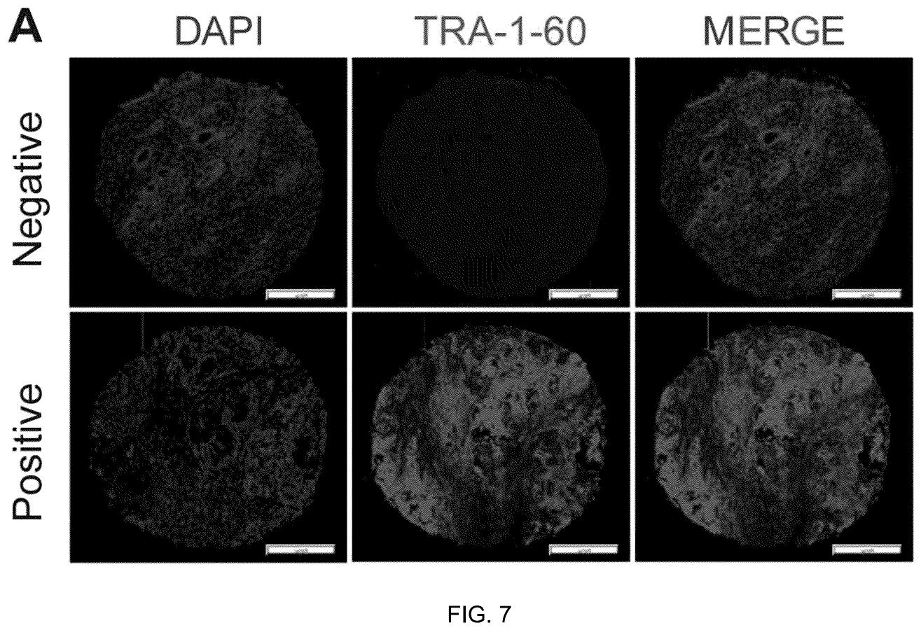

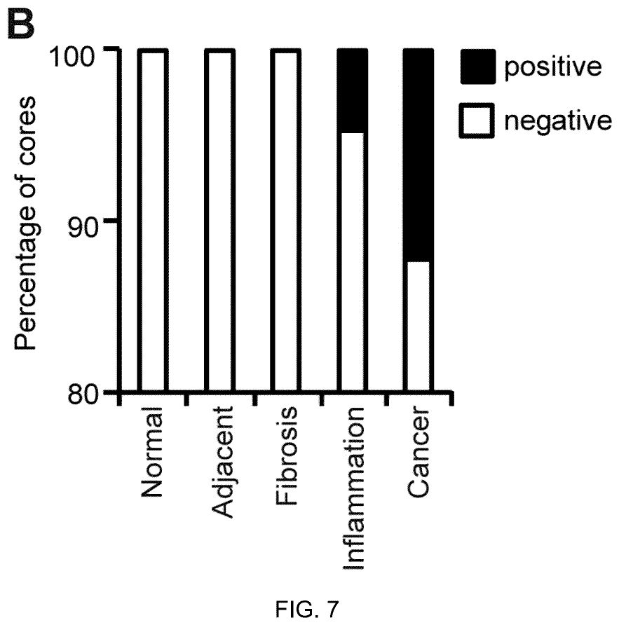

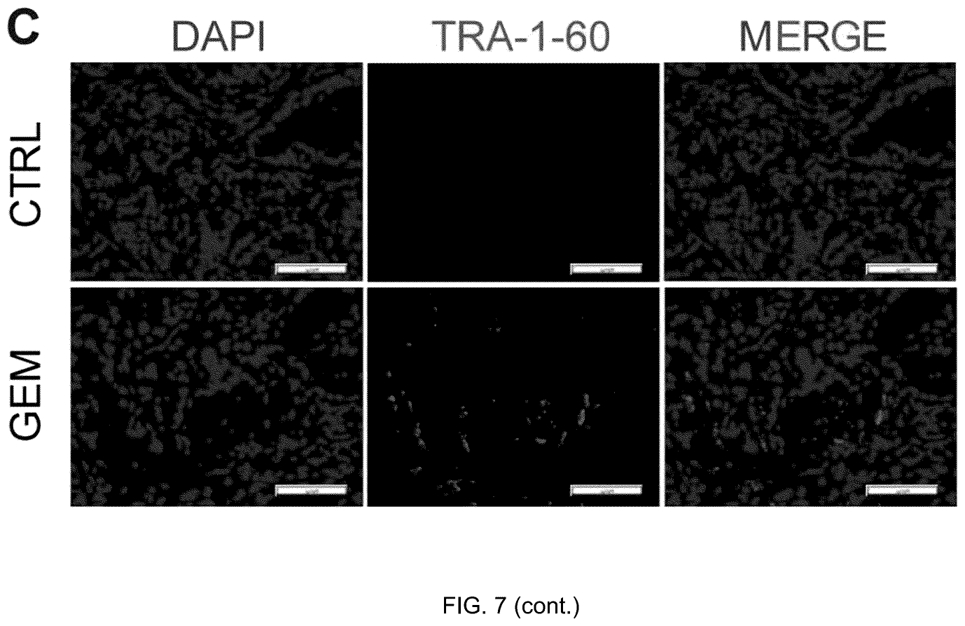

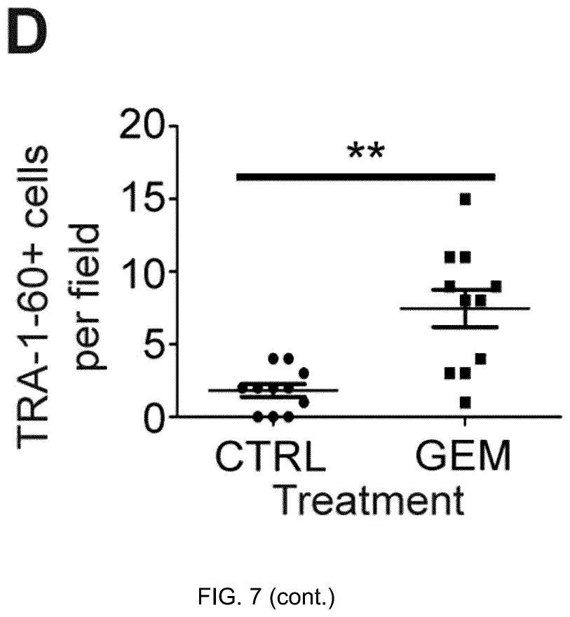

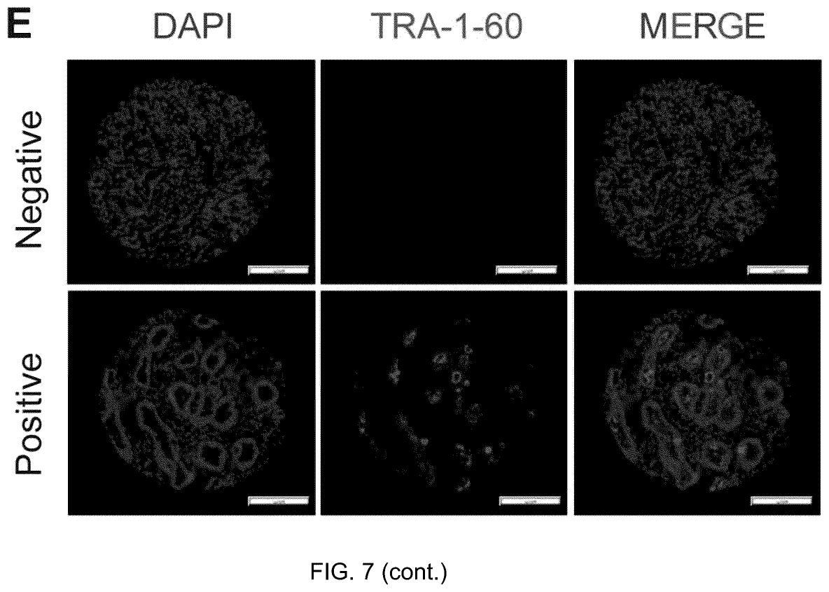

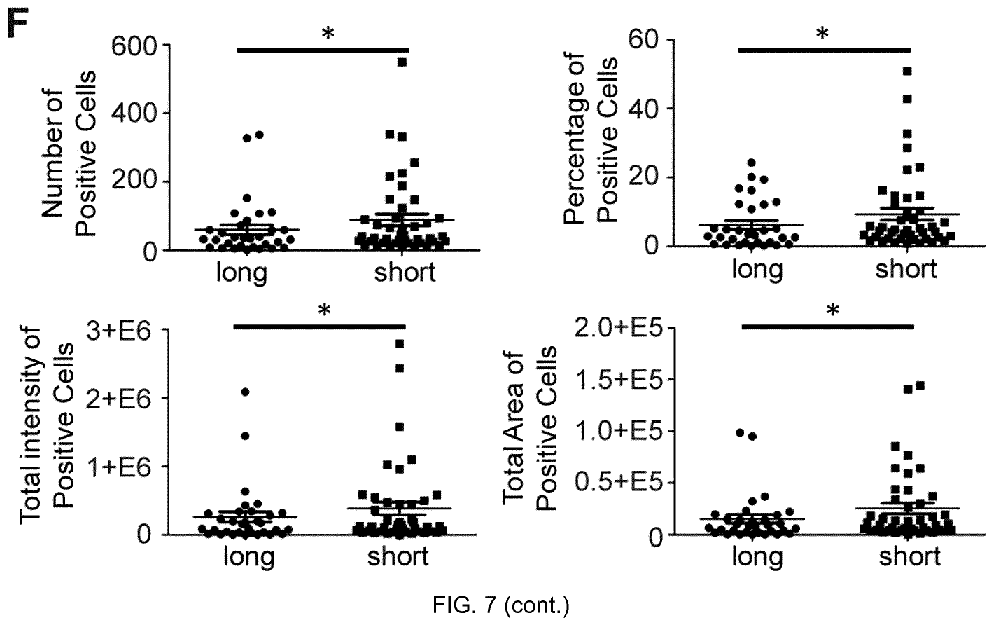

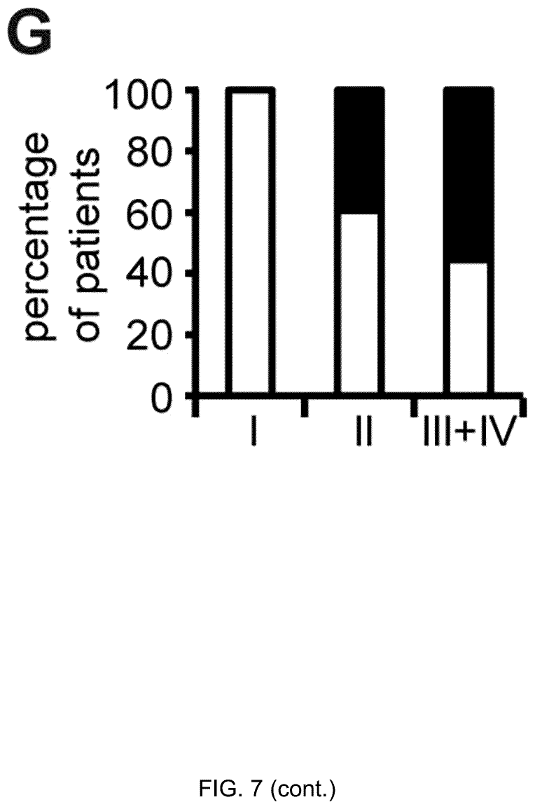

TRA-1-60/TRA-1-81 expression is positively correlated with short survival and chemoresistance of cancer patients. A primary tissue array containing pancreatic tissues with defined health conditions was stained with TRA-1-60 antibody. Cores were classified into positive or negative groups (FIG. 7A). TRA-1-60⁺ cells were detected in 12% of cancerous cores, in less than 5% of inflammation cores, and undetectable in other types (normal, fibrosis and cancer-adjacent) (FIG. 7B), which further validates the specificity of TRA-1-60 expression.

To examine whether TRA-1-60 expression correlates with chemotherapy treatment in primary tumors, a human pancreatic adenocarcinoma specimen was freshly collected after surgery, transplanted to nude mice and expanded subcutaneously. Mice carrying the primary human xenograft tumor were separated into two groups: one group was treated with 50 mg/kg GEM twice weekly while the other group received the same volume of PBS as control. After one month, xenograft tumors were stained with TRA-1-60 antibody. TRA-1-60 staining was detected in GEM-treated xenograft tumors but not control xenograft tumors (FIG. 7C, D), confirming that TRA-1-60 expression is positively correlated with chemoresistance.

Finally, additional tissue arrays containing 297 cores of pancreatic cancer specimens (99 patients, 3 cores for each patient) were stained with TRA-1-60 antibody (FIG. 7E). Based on patient survival history, the samples were separated into short survival and long survival groups. 32.7% (17 out of 52) long survival patients and 49.9% (22 out of 45) short survival patients stained positively with TRA-1-60 antibody. The positively stained cores was further analyzed. The number of TRA-1-60⁺ cells, the percentage of TRA-1-60⁺ cells, the total TRA-1-60 staining intensity and total TRA-1-60⁺ areas in short survival patients were significantly higher than those of long survival patients (FIG. 7F). The correlation between patients with TRA-1-60⁺ cores and their disease progression stage was analyzed (FIG. 7G). TRA-1-60⁺ cores were not detected in patients with stage I tumors, but detectable among patients with stage II III, and IV disease. An increasing trend was found in the percentage of TRA-1-60⁺ patients along the stage progression.

Discussion Identification of a specific cell surface marker can benefit targeted chemo- or immuno-therapies. From an antibody-based cell surface marker screen, the common epitope of TRA-1-60/TRA-1-81 was identified as highly enriched in CCCs. TRA-1-60 and TRA-1-81 antibodies are commonly used to label human pluripotent stem cells. Expression of their epitope decreases sharply during stem cell differentiation. TRA-1-60/TRA-1-81 expression is detected in pancreatic cancer tissues and a small portion of inflamed pancreatic tissue, but not in healthy pancreatic tissue. Considering the specificity of TRA-1-60 and TRA-1-81, minimal side effects can be expected for their potential use in targeted therapies.

The common epitope of TRA-1-60 and TRA-1-81 antibodies is an O-glycan structure present in podocalyxin. Sialofucosylated podocalyxin was found to be expressed by metastatic pancreatic cancer cells as a functional ligand of E- and L-selectin. In addition, TRA-1-60 was used in a combination of three markers to enrich tumor-initiating cells in human prostate cancer. Here, a previously unknown role of TRA-1-60 and TRA-1-81 in chemoresistance of multiple cancer types is described. In addition, TRA-1-60⁺/TRA-1-81⁺ cells showed high viability, low apoptosis, and strong capacity of colony and sphere forming in the presence of GEM. The correlation of TRA-1-60 expression with poor survival and chemoresistance of pancreatic cancer patients suggests value for using TRA-1-60/TRA-1-81 as a marker to predict pancreatic cancer patient survival and response to chemotherapy.

Although TRA-1-60 and TRA-1-81 have been used as pluripotent markers for many years, the mechanism controlling the expression of this antigen is largely unknown. Through global gene profiling, UGT1A10 was identified as a gene that is highly upregulated in both TRA-1-81⁺ cells and CCCs. Gene knockout and over-expression suggest that UGT1A10 lies within a pathway controlling TRA-1-60 antigen expression. UGT1A10 is a member of the UGT1A cluster, which encodes a family of nine UDP-glucuronosyltransferases (UGTs) to facilitate cellular detoxification and remove aromatic amines. The nine UGTs have unique alternative first exons followed by a set of common exons 2-5. Some members of this cluster have been shown to be involved in drug metabolism in liver and kidney through transferring glucuronic acid to scavenge chemicals. This gene cluster was reported to regulate chemosensitivity to MTX in breast cancer cell lines. Furthermore, UGT1A10 was indicated to facilitate detoxification of CPT-11/SN38 in colorectal cancer and lung cancer; however, its role in pancreatic cancer remains unknown. Using sgRNA-based gene knockout and overexpression experiments, it was shown that UGT1A10 is both essential and sufficient to control TRA-1-60/TRA-1-81 expression, as well as chemoresistance. Currently, the mechanism how UGT1A10 impacts chemosensitivity, as well as TRA-1-60/TRA-1-81, remains uncertain. Regardless, these results provide novel insight into the mechanism controlling expression of TRA-1-60/TRA-1-81 epitope, and provided a platform to identify novel agents that eliminate CCCs.

CYM, a cardiac glycoside, was discovered as a compound able to specifically eliminate TRA-1-60⁺/TRA-1-81⁺ cells. CYM blocks tumor cell growth and decreases chemoresistance both in vitro and in vivo. Interestingly, a previous screen indicated CYM as a differentiation inducer of hESCs, suggesting a conserved mechanism controlling TRA-1-60/TRA-1-81 expression in hESCs and CCCs. Furthermore, it was found that CYM decreases the levels of UGT1A10 transcripts, which might also contribute to its role in reducing chemoresistance. Thus, CYM and possibly other cardiac glycosides might be promising candidate drugs for combinational chemotherapy.

In conclusion, a novel cell surface marker, the epitope of TRA-1-60 and TRA-1-81 antibodies, labeling CCCs with an expression pattern restricted to malignant pancreatic tissues, was identified. This marker is an excellent candidate for antigen-specific targeted therapy. Genetic approaches were used to identify UGT1A10 as one potential regulator controlling TRA-1-60/TRA-1-81 expression and chemoresistance in cancer cells, which can be used as a new target for drug discovery. Finally, a high content chemical screen identified CYM, a cardiac glycoside, which represses UGT1A10 expression, eliminates TRA-1-60⁺/TRA-1-81⁺ cells, and increases chemosensitivity. UGT1A10 potentially regulates the generation of the “sternness” antigens and presumably other targets that impact chemoresistance. By blocking expression of UCT1A10, CYM enhances chemosensitivity leading to more potent action for GEM. CYM and its analogs may be considered for combination chemotherapy to achieve better outcomes for pancreatic cancer patients.

Materials and methods. Drugs. Gemcitabine hydrochloride (GEM), Cymarine (CYM), Irinotecan hydrochloride (CPT-11) and Pemetrexed disodium heptahydrate (PMX) were purchased from Sigma-Aldrich.

Cell Lines. Pancreatic cancer cell lines, SW1990, AsPC-1, HPAF-II, and CaPan-2, were purchased from American Type Culture Collection. Lung cancer cell line A549 was from Dr. Dingcheng Gao. Prostate cancer cell line DU145 was from Dr. Marco Seandel. Colon carcinoma cell line RKO, colorectal carcinoma cell lines HT29 and HCT116 were from Dr. Steven Lipkin. All cell lines were maintained in DMEM/F-12 media (Corning) plus 10% fetal bovine serum (Corning).

To establish GR cell lines, cells were first cultured with 10 nM GEM till cell growth became stable. And then GEM concentration was progressively increased to 20, 50 and 100 nM. Once cell growth became stable in 100 nM GEM, cells were expanded in 100 nM GEM and used in assays.

Antibody-based cell surface marker screening. SW1990 cell line was labeled with EBFP for screening. SW1990EBFP cells were mixed with SW1990GR cells in equal amount and stained with BD Lyoplate Human Cell Surface Marker Screening Panel according to instruction of manufacturer. Stained cells were analyzed in BD FACS Calibur cell analyzer controlled by AMS 1.0.2 software (Cytek). Data was quantified with FlowJo 9.8 software.

Flow cytometry. TRA-1-60 PerCP-efluor710 (eBioscience, 1:50) and TRA-1-81 PE (eBioscience, 1:100) were used. Cells were blocked with 5% FBS plus mouse IgM isotype control antibody (eBioscience, 1:100) at room temperature for 15 min, then stained on ice for 30 min. Stained cells were resuspended in PBS containing 300 nM DAPI and proceeded to analysis or sorting. Cell apoptosis was analyzed with FITC-AnnexinV labeling reagent (BD). Flow cytometry analysis was carried out on BD FACS Calibur with FlowJo 9.8 software. Sorting was performed with FACSVantage SE (Becton Dickinson).

Immunofluorescent staining. Primary antibodies were TRA-1-60 antibody (BD, 1:200), biotin conjugated TRA-1-60 antibody (eBioscience, 1:200). Secondary antibodies were donkey anti-mouse Alexa-efluor594 (Life technologies, 1:500) or streptavidin conjugated Alexa Fluor® 594 (Life technologies, 1:200).

Cells were fixed with 10% formalin for 20 min at room temperature. After 1 hour blocking in PBS with 5% horse serum and 0.3% triton-X100, primary antibodies were added to cells and incubated overnight at 4° C. Secondary antibody incubation was 1.5 hours at room temperature.

Tissue samples were fixed with 4% paraformaldehyde overnight at 4° C., washed with 70% ethanol and then embedded in paraffin. After De-paraffin and rehydration, antigen retrieval was preceded in hot antigen retrieving buffer with a normal-pressure steamer for 30 min. blocking was proceeded 1 hour at room temperature in PBS containing 1% horse serum. Primary antibody was stained overnight at 4° C. in PBS plus 0.5% Triton X-100 and 5% horse serum. Secondary antibody was incubated at room temperature for 1.5 hours.

Gene expression profiling. Total RNA samples were extracted from sorted cells with Absolutely RNA Nanoprep kit (Agilent Technologies), or from cells in culture with miRNeasy plus kit (Qiagen). RNA quality was validated with bioanalyzer (Agilent). cDNA libraries were generated using TruSeq RNA Sample Preparation kit (Illumina) and sequenced with single-reads in HiSeq2500 (Illumina). Gene expression level was analyzed using Cufflinks. Heat maps were then generated by heatmap.2 in R gplots package. Pathway enrichment analysis was performed online with DAVID functional annotation tool (https://david.ncifcrf.gov/).

Chemical library screening. The Prestwick library of FDA approved drugs (˜1280 chemicals) plus a collection of drugs in clinical trials was used for the screen on SW1990GR cells.

For screening, SW1990GR cells were plated in 384-well plates (5000 cells/well). After overnight incubation, chemicals were added at 1 μM or 10 μM in duplicate. After 5 days of treatment, plates were stained with TRA-1-60 antibody and analyzed with MetaXpress High Content Image Acquisition and Analysis System (Molecular Devices). Wells treated with DMSO were set as control. The reduction was calculated by subtracting the percentage of TRA-1-60⁺ cells in the chemical-treated wells from that of control wells. Standard deviation of TRA-1-60⁺ percentage in control wells was calculated and 3-fold of this value was taken as threshold to pick up the primary hit.

Mouse Procedures. To assess in vivo effects of Cymarin (CYM), Luciferase-labelled SW1990 cells were transplanted to nude mice subcutaneously. Each mouse received four injections (5×10⁵ cells/site). 12 days later, mice were randomly separated into 4 groups and treated with PBS (control), GEM (50 mg/kg bodyweight), CYM (2 mg/kg bodyweight) or GEM (50 mg/kg bodyweight) plus CYM (2 mg/kg bodyweight) twice weekly via intraperitoneal injection. Image acquisition was performed twice weekly with Xtreme Optical and X-ray small animal imaging system. Luciferin potassium salt (Regis), substrate of luciferase, was applied at 2 mg/25 g bodyweight in 150 μl PBS. Isoflurane (Henry Schein) was used for anesthesia during imaging.

Tissue Microarray (TMA). The TMA containing normal, non-cancer disease and cancerous human pancreatic tissues were purchased from Biomax. Another lab-fabricated TMA includes 97 cases (52 long-term and 45 short-term survival) collected from the archives of Memorial Sloan Kettering Cancer Center, which are pathologically confirmed as PDAC. Three sections from different areas of each tumor were collected. Five normal pancreatic blocks were chosen as controls. The percentages of missing cores for long and short term survival groups were indicated by H&E staining as 14% (22 out of 156) and 2% (3 out of 135), respectively.

Statistical Analysis. Statistical significance was determined by Student's t test (one-tailed) or ANOVA (two-way). All significance was defined at the p*<0.05 or p**<0.01.

Constructs. pLV-EBFP2-nuc plasmid (Addgene #36085) was used to label SW1990 cells for cell surface marker screening.

LentiLuc-blast and LentiUGT1A10-blast plasmid were generated based on LentiCas9-blast vector (Addgene, 52926). Luciferasec DNA was cloned from pGL3-promoter plasmid (Promega) with PCR amplification and inserted between AgeI and BamHI sites. UGT1A10 cDNA was PCR amplified from plasmid purchased from Dharmacon (MHS6278-202756114) and inserted between XbaI and BamHI sites. Primers were used as below:

Luc-Forward:
(SEQ ID NO: 1)
accggtATGGAAGACGCCAAAAACATAAAG
Luc-Reverse:
(SEQ ID NO: 2)
ggatccCACGGCGATCTTTCCGCCCTTCTT
UGT1A10-Forward:
(SEQ ID NO: 3)
tctagaATGGCTCGCGCAGGGTGGA
UGT1A10-Reverse:
(SEQ ID NO: 4)
agatctATGGGTCTTGGATTTGTGGGCTTT

Plasmids for knockout were cloned based on LentiCRISPRv2 vector (Addgene #52961) between two BsmBI sites (NEB, R0580S) with online guidance (https://www.addgene.org/static/data/plasmids/52/52961/52961-attachment_B3xTwla0bkYD.pdf). The sequence of sgRNAs are listed below:

CCL26-1:
(SEQ ID NO: 5)
CAGTTCCAAGGTGGAGACTC
CCL26-2:
(SEQ ID NO: 6)
TTCCAAGGTGGAGACTCAGG
KRT6b-1:
(SEQ ID NO: 7)
GGAACGGGGTCGTCTGGACT
KRT6b-2:
(SEQ ID NO: 8)
AGGCAGCTGGACAACATCGT
NFASC-1:
(SEQ ID NO: 9)
GTCAGCGAAGGATCACATCG
NFASC-2:
(SEQ ID NO: 10)
TCAATCAGGATGTTATCACG
SLC2A9-1:
(SEQ ID NO: 11)
CTACGGCTACAACCTGTCGG
SLC2A9-2:
(SEQ ID NO: 12)
TGGGGGCATTCACCACCGAC
UGT1a10-1:
(SEQ ID NO: 13)
TGGTTTTCGCCCATGCTCAA
UGT1a10-2:
(SEQ ID NO: 14)
CTATTAATGAGTTCATCCAG
UGT1a6-1:
(SEQ ID NO: 15)
GAGGTATCAACTGTAAGAAG
UGT1a6-2:
(SEQ ID NO: 16)
ACCTCCAATGAAGACCATGT
ZNF704-1:
(SEQ ID NO: 17)
GAGGACTTAAAACGTGACTG
ZNF704-2:
(SEQ ID NO: 18)
TGCTGGAGGAACATCAATGT
CTRL1:
(SEQ ID NO: 19)
GGGCGAGGAGCTGTTCACCG
CTRL2:
(SEQ ID NO: 20)
GAGCTGGACGGCGACGTAAA

Cell viability, colony formation, sphere formation and growth curve. To quantify viability, cells were plated into 96-well plates (6000/well) one night before treatment. Drugs were then added in indicated dose for 5 days. After fixation, cell nuclei were stained with DAPI (Invitrogen). Plates were analyzed using MetaXpress High Content Image Acquisition and Analysis System (Molecular Devices).

For colony formation, cells were seeded to 6-well plates (2000/well) with or without GEM. Medium was refreshed every 2 days. Four days later, GEM-free media was applied to all wells for another 4 days of culture. The plates were then stained with 20 mg/ml crystal violet (SigmaAldrich) for 30 min. After washing with running water, the colony numbers were counted.

For sphere formation, cells were plated to ultra-low attach 6-well plate (2000/well) in DMEM/F-12 supplemented with 1× B27, 20 ng/ml bFGF and 20 ng/ml EGF plus given dose of GEM. 8 days later, number of spheres was counted under a microscope.

To quantify growth, Cells were seeded into 96-well plates (2000/well). After overnight incubation, medium with indicated drug was refreshed. Cell proliferation was quantified every 2 days using CellTiter 96® AQueous Non-Radioactive Cell Proliferation Assay (Promega).

qRT-PCR. Reverse transcription reaction was prepared with 0.5-2 μg total RNA using High Capacity cDNA Reverse Transcription Kit (Applied Biosystems). Products were diluted 10 folds with DNase&RNase free water as template. qPCR reactions were prepared with 2× LightCycler 480 SYBR Green I Master reagent (Roche) and proceeded in LightCycler 480 II (Roche) with software LightCycler 480 SW1.5.1 for signal acquisition and processing. ACTB was employed as internal reference. Primers were listed below:

UGT1A10-Forward:
(SEQ ID NO: 21)
GACTATCCCAAACCCGTGATG
UGT1A10-Reverse:
(SEQ ID NO: 22)
CCCAAAGAGAAAACCACAATTCC
ACTB-Forward:
(SEQ ID NO: 23)
ACCTTCTACAATGAGCTGCG
ACTB-Reverse:
(SEQ ID NO: 24)
CCTGGATAGCAACGTACATGG

Western blot. Cells were lyzed in complete RIPA buffer (Millipore). Lysates were loaded on 4-12% Bis-Tris gels (Life technologies). After electrophoresis, protein was transferred to nitrocellulose membrane (Life technologies) and blocked with 5% milk in TBST, then incubated with primary antibody overnight. The antibodies are mouse anti-UGT1A10 polyclonal antibody (Abcam, abl94474) and mouse anti-GAPDH antibody (Abcam, ab8245). The membrane was washed and incubated with secondary antibody, peroxidase Horse Anti-Mouse IgG Antibody (Vector Labs), in TBST containing 5% milk for 1 hour at room temperature. Membrane was developed with Chemiluminescent Substrate (LI-COR) and scanned with C-DiGit Blot Scanner (Licor) and analyzed with Image Studio Digits 4.0 software.

Although the present disclosure has been described with respect to one or more particular example(s), it will be understood that other examples of the present disclosure may be made without departing from the scope of the present disclosure.

1. A pharmaceutical composition comprising Cymarin and/or a structural analog or analogs thereof and/or one or more pharmaceutically acceptable salt, a salt, a partial salt, a solvate, a hydrate, a polymorph, or a prodrug thereof and/or a stereoisomer or a mixture of stereoisomers, an isotopic variant, or a tautomer thereof.

2. The composition of claim 1, wherein the pharmaceutical composition further comprises one or more pharmaceutically acceptable excipient(s).

3. A method of downregulating of UGT1A10, at least partially eliminating TRA-1-60 and/or TRA-1-81 expression, or increasing chemosensitivity of chemoresistant cancer cells (CCCs), if present, or any combination thereof in a subject comprising:
administering to a subject an effective amount of Cymarin and/or a structural analog or analogs thereof and/or one or more pharmaceutically acceptable salt, a salt, a partial salt, a solvate, a hydrate, a polymorph, a prodrug of the Cymarin and/or the structural analog(s) and/or a stereoisomer or a mixture of stereoisomers, an isotopic variant, or a tautomer, of the Cymarin and/or the structural analog(s) and/or one or more pharmaceutical composition(s) of claim 1, wherein UGT1A10 is downregulated, TRA-1-60 and/or TRA-1-81 expression is at least partially eliminated, or chemosensitivity of chemoresistant cancer cells (CCCs), if present, or any combination thereof is increased in the subject.

4. The method of claim 3, wherein UGT1A10 is downregulated.

5. The method of claim 3, wherein TRA-1-60 and/or TRA-1-81 expression is at least partially eliminated.

6. The method of claim 5, wherein TRA-1-60 and/or TRA-1-81 expression is completely eliminated.

7. The method of claim 3, wherein chemosensitivity of CCCs is increased.

8. A method for treating one or more cancer(s), wherein each cancer is characterized by TRA-1-60 and TRA-1-81 enrichment in chemosensitivity of chemoresistant cancer cells (CCCs), if present, or a presence of TRA-1-60⁺/TRA-1-81⁺ cells, or both, in a subject comprising:
administering to subject diagnosed with or in need of treatment for the cancer(s) an effective amount of Cymarin and/or a structural analog or analogs thereof and/or one or more pharmaceutically acceptable salt, a salt, a partial salt, a solvate, a hydrate, a polymorph, a prodrug of the Cymarin and/or structural analogs and/or a stereoisomer or a mixture of stereoisomers, an isotopic variant, or a tautomer, of the Cymarin and/or structural analog(s) and/or one or more pharmaceutical composition(s) of claim 1 that results in downregulation of UGT1A10, at least partially eliminating TRA-1-60 and/or TRA-1-81 expression, increasing chemosensitivity of chemoresistant cancer cells (CCCs), if present, or any combination thereof.

9. The method of claim 8, wherein UGT1A10 is downregulated.

10. The method of claim 8, wherein TRA-1-60 and/or TRA-1-81 expression is at least partially eliminated.

11. The method of claim 10, wherein TRA-1-60 and/or TRA-1-81 expression is completely eliminated.

12. The method of claim 8, wherein chemosensitivity of CCCs is increased.

13. The method of claim 8, wherein cancer(s) is/are pancreatic cancer, colon cancer, colorectal cancer, lung cancer, breast cancer, prostate cancer, or any combination thereof.

14. A method of treating a subject diagnosed with or is in need of treatment for pancreatic cancer, colon cancer, colorectal cancer, lung cancer, breast cancer, prostate cancer, or any combination thereof comprising
administering to a subject an effective amount of Cymarin and/or a structural analog or analogs thereof and/or one or more pharmaceutically acceptable salt, a salt, a partial salt, a solvate, a hydrate, a polymorph, a prodrug of the Cymarin and/or the structural analog(s) and/or a stereoisomer or a mixture of stereoisomers, an isotopic variant, or a tautomer, of the Cymarin and/or the structural analog(s) and/or one or more pharmaceutical composition(s) of claim 1, wherein one or more symptom(s) and/or indication(s) of the subject is at least partially alleviated.

15. The method of claim 14, wherein the UGT1A10 is downregulated, TRA-1-60 and/or TRA-1-81 expression is at least partially eliminated, or, in the case where one or more of the pancreatic cancer, the colon cancer, the colorectal cancer, the lung cancer, the breast cancer, the prostate cancer, or the combination thereof, if present, is a chemoresistant cancer, chemosensitivity of chemoresistant cancer cells (CCCs) is increased in the subject.

16. The method of claim 15, wherein UGT1A10 is downregulated.

17. The method of claim 15, wherein TRA-1-60 and/or TRA-1-81 expression is at least partially eliminated.

18. The method of claim 17, wherein TRA-1-60 and/or TRA-1-81 expression is completely eliminated.

19. The method of claim 15, wherein, in the case where one or more of the pancreatic cancer, the colon cancer, colorectal cancer, the lung cancer, the breast cancer, the prostate cancer, or the combination thereof, if present, is/are a chemoresistant cancer or cancers, chemosensitivity of CCCs is increased.

20. The method of claim 14, wherein the pancreatic cancer is chemoresistant pancreatic cancer and/or the colon cancer is chemoresistant colon cancer and/or the colorectal cancer is chemoresistant colorectal cancer and/or the lung cancer is chemoresistant lung cancer and/or the breast cancer is chemoresistant breast cancer and/or the prostate cancer is chemoresistant prostate cancer.

21. The method of claim 20, wherein one or more symptom(s) and/or indication(s) of the subject is completely alleviated.


SEQUENCE LISTING (20 sequences)

SEQ ID NO: 1 - DNA, 30 bp, synthetic construct
  source 1..30: mol_type = other DNA
accggtatggaagacgccaaaaacataaag

SEQ ID NO: 2 - DNA, 30 bp, synthetic construct
  source 1..30: mol_type = other DNA
ggatcccacggcgatctttccgcccttctt

SEQ ID NO: 3 - DNA, 25 bp, synthetic construct
  source 1..25: mol_type = other DNA
tctagaatggctcgcgcagggtgga

SEQ ID NO: 4 - DNA, 30 bp, synthetic construct
  source 1..30: mol_type = other DNA
agatctatgggtcttggatttgtgggcttt

SEQ ID NO: 5 - DNA, 20 bp, synthetic construct
  source 1..20: mol_type = other DNA
cagttccaaggtggagactc

SEQ ID NO: 6 - DNA, 20 bp, synthetic construct
  source 1..20: mol_type = other DNA
ttccaaggtggagactcagg

SEQ ID NO: 7 - DNA, 20 bp, synthetic construct
  source 1..20: mol_type = other DNA
ggaacggggtcgtctggact

SEQ ID NO: 8 - DNA, 20 bp, synthetic construct
  source 1..20: mol_type = other DNA
aggcagctggacaacatcgt

SEQ ID NO: 9 - DNA, 20 bp, synthetic construct
  source 1..20: mol_type = other DNA
gtcagcgaaggatcacatcg

SEQ ID NO: 10 - DNA, 20 bp, synthetic construct
  source 1..20: mol_type = other DNA
tcaatcaggatgttatcacg

SEQ ID NO: 11 - DNA, 20 bp, synthetic construct
  source 1..20: mol_type = other DNA
ctacggctacaacctgtcgg

SEQ ID NO: 12 - DNA, 20 bp, synthetic construct
  source 1..20: mol_type = other DNA
tgggggcattcaccaccgac

SEQ ID NO: 13 - DNA, 20 bp, synthetic construct
  source 1..20: mol_type = other DNA
tggttttcgcccatgctcaa

SEQ ID NO: 14 - DNA, 20 bp, synthetic construct
  source 1..20: mol_type = other DNA
ctattaatgagttcatccag

SEQ ID NO: 15 - DNA, 20 bp, synthetic construct
  source 1..20: mol_type = other DNA
gaggtatcaactgtaagaag

SEQ ID NO: 16 - DNA, 20 bp, synthetic construct
  source 1..20: mol_type = other DNA
acctccaatgaagaccatgt

SEQ ID NO: 17 - DNA, 20 bp, synthetic construct
  source 1..20: mol_type = other DNA
gaggacttaaaacgtgactg

SEQ ID NO: 18 - DNA, 20 bp, synthetic construct
  source 1..20: mol_type = other DNA
tgctggaggaacatcaatgt

SEQ ID NO: 19 - DNA, 20 bp, synthetic construct
  source 1..20: mol_type = other DNA
gggcgaggagctgttcaccg

SEQ ID NO: 20 - DNA, 20 bp, synthetic construct
  source 1..20: mol_type = other DNA
gagctggacggcgacgtaaa